(19) United States
(12) Patent Application Publication
(10) Pub. No.: US 2026/0223937 A1
(43) Pub. Date: Aug. 6, 2026

(54) AEROSOL DELIVERY SYSTEMS AND METHODS

(71) Applicant: Nicoventures Trading Limited, London (GB)

(72) Inventor: Ruifan LI, Shenzhen (CN)

(21) Appl. No.: 19/153,146

(22) PCT Filed: Jan. 30, 2024
(86) PCT No.: PCT/GB2024/050246

§ 371 Date: Aug. 1, 2025

(30) Foreign Application Priority Data
Feb. 3, 2023 (CN) 202310092884.7
May 17, 2023 (GB) 2307357.0

(51) Int. Cl. A24F 40/485; A24F 7/00; A24F 40/42; A24F 40/51; A24F 40/70; A61M 15/00; A61M 15/06

(52) U.S. Cl. CPC A24F 40/485; A24F 7/00; A24F 40/42; A24F 40/51; A24F 40/70; A61M 15/002; A61M 15/06; A61M 2207/00

(57) ABSTRACT

An aerosol delivery subsystem for adjusting fluid flow in an aerosol delivery system, comprising: an air inlet; a baffle assembly having an aperture therethrough; and an engagement mechanism for positively engaging the baffle assembly with respect to the subsystem in at least one of a plurality of predetermined positions as the baffle assembly is moved with respect to the air inlet, wherein different ones of the plurality of predetermined positions result in different degrees of alignment between the baffle assembly aperture and the air inlet, to alter air flow through the air inlet.

### CROSS-REFERENCE TO RELATED APPLICATIONS

This present application is a National Phase entry of PCT Application No. PCT/GB2024/050246, filed 30 Jan. 2024, which claims priority from Great Britain Application No. 2307357.0, filed 17 May 2023, and which claims priority from Chinese Application No. 202310092884.7, filed 3 Feb. 2023, each of which are fully incorporated herein by reference in their entireties.

### FIELD

The present disclosure relates to aerosol delivery systems such as, but not exclusively, nicotine delivery systems (e.g. e-cigarettes).

### BACKGROUND

Aerosol delivery systems such as electronic cigarettes (e-cigarettes) generally contain an aerosol generating material, such as a chamber of a source solid or liquid, which may contain an active substance and/or a flavour, from which an aerosol or vapour is generated for inhalation by a user, for example through heat vaporisation. Thus, an aerosol delivery system will typically comprise an aerosol generation area containing an aerosol generator, e.g. a heating element, arranged to vaporise or aerosolise a portion of precursor material to generate a vapour or aerosol in the aerosol generation area. As a user inhales on the device and electrical power is supplied to the vaporiser, air is drawn into the device through an inlet hole and along an inlet air channel connecting to the aerosol generation area, where the air mixes with vaporised precursor material to form a condensation aerosol. There is an outlet channel connecting the aerosol generation area to an outlet in the mouthpiece and the air drawn into the aerosol generation area as a user inhales on the mouthpiece continues along the outlet flow path to the mouthpiece outlet, carrying the aerosol with it, for inhalation by the user. Some electronic cigarettes may also include a flavour element in the air flow path through the device to impart additional flavours. Such devices may sometimes be referred to as hybrid devices, and the flavour element may, for example, include a portion of tobacco arranged in the air flow path between the aerosol generation area and the mouthpiece such that aerosol/condensation aerosol drawn through the device passes through the portion of tobacco before exiting the mouthpiece for user inhalation.

WO 2016/012774, incorporated herein by reference, discloses electronic vapour provision systems comprising a collar around a housing for a user to adjust alignment between one or more air inlet holes of the housing and the collar, to provide different levels of ventilation to the system.

WO 2017/046566, incorporated herein by reference, discloses an aerosol provision system with an airflow adjuster in the airflow path, downstream from the air inlet.

User experiences with electronic aerosol delivery systems are continually improving as such systems become more refined in respect of the nature of the vapour they provide for user inhalation, for example in terms of deep lung delivery, mouth feel and consistency in performance. Nonetheless, approaches for improving further still on these aspects of electronic vapour provision systems remain of interest. In particular, it is of interest to develop approaches in which an aerosol delivery system comprises functionality enabling operating characteristics of the system to be adjusted, to target certain operating characteristics which may be desirable to a user.

Various approaches are described herein which seek to help address or mitigate at least some of the issues discussed above.

### Terminology

### Delivery System

As used herein, the term “delivery system” is intended to encompass systems that deliver at least one substance to a user in use, and includes:
combustible aerosol provision systems, such as cigarettes, cigarillos, cigars, and tobacco for pipes or for roll-your-own or for make-your-own cigarettes (whether based on tobacco, tobacco derivatives, expanded tobacco, reconstituted tobacco, tobacco substitutes or other smokable material);
non-combustible aerosol provision systems that release compounds from an aerosol-generating material without combusting the aerosol-generating material, such as electronic cigarettes, tobacco heating products, and hybrid systems to generate aerosol using a combination of aerosol-generating materials; and
aerosol-free delivery systems that deliver the at least one substance to a user orally, nasally, transdermally or in another way without forming an aerosol, including but not limited to, lozenges, gums, patches, articles comprising inhalable powders, and oral products such as oral tobacco which includes snus or moist snuff, wherein the at least one substance may or may not comprise nicotine.

### Combustible Aerosol Provision System

According to the present disclosure, a “combustible” aerosol provision system is one where a constituent aerosol-generating material of the aerosol provision system (or component thereof) is combusted or burned during use in order to facilitate delivery of at least one substance to a user.

In some embodiments, the delivery system is a combustible aerosol provision system, such as a system selected from the group consisting of a cigarette, a cigarillo and a cigar. In some embodiments, the disclosure relates to a component for use in a combustible aerosol provision system, such as a filter, a filter rod, a filter segment, a tobacco rod, a spill, an aerosol-modifying agent release component such as a capsule, a thread, or a bead, or a paper such as a plug wrap, a tipping paper or a cigarette paper.

### Non-combustible Aerosol Provision System

According to the present disclosure, a “non-combustible” aerosol provision system is one where a constituent aerosol-generating material of the aerosol provision system (or component thereof) is not combusted or burned in order to facilitate delivery of at least one substance to a user.

In some embodiments, the delivery system is a non-combustible aerosol provision system, such as a powered non-combustible aerosol provision system. In some embodiments, the non-combustible aerosol provision system is an electronic cigarette, also known as a vaping device or electronic nicotine delivery system (END), although it is noted that the presence of nicotine in the aerosol-generating material is not a requirement. In some embodiments, the non-combustible aerosol provision system is an aerosol-generating material heating system, also known as a heat-not-burn system. An example of such a system is a tobacco heating system.

In some embodiments, the non-combustible aerosol provision system is a hybrid system to generate aerosol using a combination of aerosol-generating materials, one or a plurality of which may be heated. Each of the aerosol-generating materials may be, for example, in the form of a solid, liquid or gel and may or may not contain nicotine. In some embodiments, the hybrid system comprises a liquid or gel aerosol-generating material and a solid aerosol-generating material. The solid aerosol-generating material may comprise, for example, tobacco or a non-tobacco product.

Typically, the non-combustible aerosol provision system may comprise a non-combustible aerosol provision device and a consumable for use with the non-combustible aerosol provision device. In some embodiments, the disclosure relates to consumables comprising aerosol-generating material and configured to be used with non-combustible aerosol provision devices. These consumables are sometimes referred to as articles throughout the disclosure.

In some embodiments, the non-combustible aerosol provision system, such as a non-combustible aerosol provision device thereof, may comprise a power source and a controller. The power source may, for example, be an electric power source or an exothermic power source. In some embodiments, the exothermic power source comprises a carbon substrate which may be energised so as to distribute power in the form of heat to an aerosol-generating material or to a heat transfer material in proximity to the exothermic power source.

In some embodiments, the non-combustible aerosol provision system may comprise an area for receiving the consumable, an aerosol generator, an aerosol generation area, a housing, a mouthpiece, a filter and/or an aerosol-modifying agent. In some embodiments, the consumable for use with the non-combustible aerosol provision device may comprise aerosol-generating material, an aerosol-generating material storage area, an aerosol-generating material transfer component, an aerosol generator, an aerosol generation area, a housing, a wrapper, a filter, a mouthpiece, and/or an aerosol-modifying agent.

### Aerosol-Free Delivery System

In some embodiments, the delivery system is an aerosol-free delivery system that delivers at least one substance to a user orally, nasally, transdermally or in another way without forming an aerosol, including but not limited to, lozenges, gums, patches, articles comprising inhalable powders, and oral products such as oral tobacco which includes snus or moist snuff, wherein the at least one substance may or may not comprise nicotine.

In some embodiments, the substance to be delivered may be an aerosol-generating material or a material that is not intended to be aerosolised. As appropriate, either material may comprise one or more active constituents, one or more flavours, one or more aerosol-former materials, and/or one or more other functional materials.

### Active Substance

In some embodiments, the substance to be delivered comprises an active substance. The active substance as used herein may be a physiologically active material, which is a material intended to achieve or enhance a physiological response. The active substance may for example be selected from nutraceuticals, nootropics, psychoactives. The active substance may be naturally occurring or synthetically obtained. The active substance may comprise for example nicotine, caffeine, taurine, theine, vitamins such as B6 or B12 or C, melatonin, cannabinoids, or constituents, derivatives, or combinations thereof. The active substance may comprise one or more constituents, derivatives or extracts of tobacco, cannabis or another botanical.

In some embodiments, the active substance comprises nicotine. In some embodiments, the active substance comprises caffeine, melatonin or vitamin B12.

As noted herein, the active substance may comprise one or more constituents, derivatives or extracts of cannabis, such as one or more cannabinoids or terpenes. As noted herein, the active substance may comprise or be derived from one or more botanicals or constituents, derivatives or extracts thereof. As used herein, the term “botanical” includes any material derived from plants including, but not limited to, extracts, leaves, bark, fibres, stems, roots, seeds, flowers, fruits, pollen, husk, shells or the like. Alternatively, the material may comprise an active compound naturally existing in a botanical, obtained synthetically. The material may be in the form of liquid, gas, solid, powder, dust, crushed particles, granules, pellets, shreds, strips, sheets, or the like.

Example botanicals are tobacco, eucalyptus, star anise, hemp, cocoa, cannabis, fennel, lemongrass, peppermint, spearmint, rooibos, chamomile, flax, ginger, ginkgo biloba, hazel, hibiscus, laurel, licorice (liquorice), matcha, mate, orange skin, papaya, rose, sage, tea such as green tea or black tea, thyme, clove, cinnamon, coffee, aniseed (anise), basil, bay leaves, cardamom, coriander, cumin, nutmeg, oregano, paprika, rosemary, saffron, lavender, lemon peel, mint, juniper, elderflower, vanilla, wintergreen, beefsteak plant, curcuma, turmeric, sandalwood, cilantro, bergamot, orange blossom, myrtle, cassis, valerian, pimento, mace, damien, marjoram, olive, lemon balm, lemon basil, chive, carvi, verbena, tarragon, geranium, mulberry, ginseng, theanine, theacrine, maca, ashwagandha, damiana, guarana, chlorophyll, baobab or any combination thereof. The mint may be chosen from the following mint varieties: Mentha arventis, Mentha c.v., Mentha niliaca, Mentha piperita, Mentha piperita citrata c.v., Mentha piperita c. v, Mentha spicata crispa, Mentha cardifolia, Memtha longifolia, Mentha suaveolens variegata, Mentha pulegium, Mentha spicata c.v. and Mentha suaveolens.

In some embodiments, the active substance comprises or is derived from one or more botanicals or constituents, derivatives or extracts thereof and the botanical is tobacco. In some embodiments, the active substance comprises or derived from one or more botanicals or constituents, derivatives or extracts thereof and the botanical is selected from eucalyptus, star anise, cocoa and hemp. In some embodiments, the active substance comprises or derived from one or more botanicals or constituents, derivatives or extracts thereof and the botanical is selected from rooibos and fennel.

### Flavours

In some embodiments, the substance to be delivered comprises a flavour. As used herein, the terms “flavour” and “flavourant” refer to materials which, where local regulations permit, may be used to create a desired taste, aroma or other somatosensorial sensation in a product for adult consumers. They may include naturally occurring flavour materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balm, lemon basil, chive, carvi, verbena, tarragon, limonene, thymol, camphene), flavour enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, liquid such as an oil, solid such as a powder, or gas.

In some embodiments, the flavour comprises menthol, spearmint and/or peppermint. In some embodiments, the flavour comprises flavour components of cucumber, blueberry, citrus fruits and/or redberry. In some embodiments, the flavour comprises eugenol. In some embodiments, the flavour comprises flavour components extracted from tobacco. In some embodiments, the flavour comprises flavour components extracted from cannabis.

In some embodiments, the flavour may comprise a sensate, which is intended to achieve a somatosensorial sensation which are usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether and a suitable cooling agent may be, but not limited to eucolyptol, WS-3.

### Aerosol-Generating Material

Aerosol-generating material is a material that is capable of generating aerosol, for example when heated, irradiated or energized in any other way. Aerosol-generating material may, for example, be in the form of a solid, liquid or gel which may or may not contain an active substance and/or flavourants. In some embodiments, the aerosol-generating material may comprise an “amorphous solid”, which may alternatively be referred to as a “monolithic solid” (i.e. non-fibrous). In some embodiments, the amorphous solid may be a dried gel. The amorphous solid is a solid material that may retain some fluid, such as liquid, within it. In some embodiments, the aerosol-generating material may for example comprise from about 50 wt %, 60 wt % or 70 wt % of amorphous solid, to about 90 wt %, 95 wt % or 100 wt % of amorphous solid.

The aerosol-generating material may comprise one or more active substances and/or flavours, one or more aerosol-former materials, and optionally one or more other functional material.

### Aerosol-Former Material

The aerosol-former material may comprise one or more constituents capable of forming an aerosol. In some embodiments, the aerosol-former material may comprise one or more of glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

### Functional Material

The one or more other functional materials may comprise one or more of pH regulators, colouring agents, preservatives, binders, fillers, stabilizers, and/or antioxidants.

### Substrate

The material may be present on or in a support, to form a substrate. The support may, for example, be or comprise paper, card, paperboard, cardboard, reconstituted material, a plastics material, a ceramic material, a composite material, glass, a metal, or a metal alloy. In some embodiments, the support comprises a susceptor. In some embodiments, the susceptor is embedded within the material. In some alternative embodiments, the susceptor is on one or either side of the material.

### Consumable

A consumable is an article comprising or consisting of aerosol-generating material, part or all of which is intended to be consumed during use by a user. A consumable may comprise one or more other components, such as an aerosol-generating material storage area, an aerosol-generating material transfer component, an aerosol generation area, a housing, a wrapper, a mouthpiece, a filter and/or an aerosol-modifying agent. A consumable may also comprise an aerosol generator, such as a heater, that emits heat to cause the aerosol-generating material to generate aerosol in use. The heater may, for example, comprise combustible material, a material heatable by electrical conduction, or a susceptor.

### Susceptor

A susceptor is a material that is heatable by penetration with a varying magnetic field, such as an alternating magnetic field. The susceptor may be an electrically-conductive material, so that penetration thereof with a varying magnetic field causes induction heating of the heating material. The heating material may be magnetic material, so that penetration thereof with a varying magnetic field causes magnetic hysteresis heating of the heating material. The susceptor may be both electrically-conductive and magnetic, so that the susceptor is heatable by both heating mechanisms. The device that is configured to generate the varying magnetic field is referred to as a magnetic field generator, herein.

### Aerosol-Modifying Agent

An aerosol-modifying agent is a substance, typically located downstream of the aerosol generation area, that is configured to modify the aerosol generated, for example by changing the taste, flavour, acidity or another characteristic of the aerosol. The aerosol-modifying agent may be provided in an aerosol-modifying agent release component, that is operable to selectively release the aerosol-modifying agent. The aerosol-modifying agent may, for example, be an additive or a sorbent. The aerosol-modifying agent may, for example, comprise one or more of a flavourant, a colourant, water, and a carbon adsorbent. The aerosol-modifying agent may, for example, be a solid, a liquid, or a gel. The aerosol-modifying agent may be in powder, thread or granule form. The aerosol-modifying agent may be free from filtration material.

### Aerosol Generator

An aerosol generator is an apparatus configured to cause aerosol to be generated from the aerosol-generating material. In some embodiments, the aerosol generator is a heater configured to subject the aerosol-generating material to heat energy, so as to release one or more volatiles from the aerosol-generating material to form an aerosol. In some embodiments, the aerosol generator is configured to cause an aerosol to be generated from the aerosol-generating material without heating. For example, the aerosol generator may be configured to subject the aerosol-generating material to one or more of vibration, increased pressure, or electrostatic energy.

The present disclosure relates to aerosol delivery systems (which may also be referred to as vapour delivery systems) such as nebulisers or e-cigarettes. Throughout the following description the term “e-cigarette” or “electronic cigarette” may sometimes be used, but it will be appreciated this term may be used interchangeably with aerosol delivery system/device and electronic aerosol delivery system/device. Furthermore, and as is common in the technical field, the terms “aerosol” and “vapour”, and related terms such as “vaporise”, “volatilise” and “aerosolise”, may generally be used interchangeably.

Aerosol delivery systems (e-cigarettes) often, though not always, comprise a modular assembly comprising a reusable device part and a replaceable (disposable/consumable) cartridge part. Often, the replaceable cartridge part will comprise the aerosol generating material and the vaporiser (which may collectively be called a ‘cartomizer’) and the reusable device part will comprise the power supply (e.g. rechargeable power source) and control circuitry. It will be appreciated these different parts may comprise further elements depending on functionality. For example, the reusable device part will often comprise a user interface for receiving user input and displaying operating status characteristics, and the replaceable cartridge device part in some cases comprises a temperature sensor for helping to control temperature. Cartridges are electrically and mechanically coupled to the control unit for use, for example using a screw thread, bayonet, or magnetic coupling with appropriately arranged electrical contacts. When the aerosol generating material in a cartridge is exhausted, or the user wishes to switch to a different cartridge having a different aerosol generating material, the cartridge may be removed from the reusable part and a replacement cartridge attached in its place. Systems and devices conforming to this type of two-part modular configuration may generally be referred to as two-part systems/devices.

It is common for electronic cigarettes to have a generally elongate shape. For the sake of providing a concrete example, certain embodiments of the disclosure will be taken to comprise this kind of generally elongate two-part system employing disposable cartridges. However, it will be appreciated that the underlying principles described herein may equally be adopted for different configurations, for example single-part systems or modular systems comprising more than two parts, refillable devices and single-use disposables, as well as other overall shapes, for example based on so-called box-mod high performance devices that typically have a boxier shape. More generally, it will be appreciated certain embodiments of the disclosure are based on aerosol delivery systems which are operationally configured to provide functionality in accordance with the principles described herein and the constructional aspects of systems configured to provide the functionality in accordance with certain embodiments of the disclosure is not of primary significance.

### BRIEF SUMMARY OF THE INVENTION

The present invention provides an aerosol delivery subsystem 100 for adjusting fluid flow in an aerosol delivery system 1, comprising: an air inlet 28; a baffle assembly 150 having an aperture 154 therethrough; and an engagement mechanism for positively engaging the baffle assembly 150 with respect to the subsystem 100 in at least one of a plurality of predetermined positions as the baffle assembly 150 is moved with respect to the air inlet 28, wherein different ones of the plurality of predetermined positions result in different degrees of alignment between the baffle assembly aperture 154 and the air inlet 28, to alter air flow through the air inlet 28.

The present invention further provides a method of manufacturing an aerosol delivery subsystem for an aerosol delivery system, comprising providing: an air inlet 28; a baffle assembly 150 having an aperture 154 therethrough; and an engagement mechanism; the method comprising configuring the engagement mechanism for positively engaging the baffle assembly 150 with respect to the subsystem 100 in at least one of a plurality of predetermined positions as the baffle assembly 150 is moved with respect to the air inlet 28, wherein different ones of the plurality of predetermined positions result in different degrees of alignment between the baffle assembly aperture 154 and the air inlet 28, to alter air flow through the air inlet 28.

The present invention further provides an aerosol delivery subsystem 100 for adjusting fluid flow in an aerosol delivery system 1, comprising a primary air inlet 28 into the subsystem 100; a secondary air inlet 128 into the subsystem 100; a fluid flow sensor 30; and a baffle assembly 150, wherein: the primary air inlet 28 provides an air flow path into the subsystem 100 via the fluid flow sensor 30; the secondary air inlet 128 provides an air flow path into the subsystem 100 bypassing the fluid flow sensor 30; and the baffle assembly 150 is movable with respect to the subsystem 100 into a plurality of positions to selectively alter air flow through one or more of the primary and secondary air inlets 28, 128.

The present invention further provides a method of manufacturing an aerosol delivery subsystem for an aerosol delivery system, comprising providing: a primary air inlet 28; a secondary air inlet 128; a fluid flow sensor 30; and a baffle assembly 150, the method comprising configuring the subsystem wherein: the primary air inlet 28 provides an air flow path into the subsystem 100 via the fluid flow sensor 30; the secondary air inlet 128 provides an air flow path into the subsystem 100 bypassing the fluid flow sensor 30; and the baffle assembly 150 is movable with respect to the subsystem 100 into a plurality of positions to selectively alter air flow through one or more of the primary and secondary air inlets 28, 128.

The present invention further provides additional embodiments as claimed in the dependent claims.

The claimed invention generally provides a sub-assembly or subsystem suitable for use in an aerosol delivery system, or configured for use in an aerosol delivery system. The subsystem may generally form part of an aerosol delivery system and in particular may form part of the reusable device and/or the consumable cartridge.

The claimed invention may advantageously provide adjustable operating characteristics to target certain characteristics which may be desirable to a user.

### BRIEF DESCRIPTION OF THE FIGURES

Embodiments of the disclosure will now be described, by way of example only, with reference to the accompanying drawings, in which:

FIG. 1 is a schematic cross-section view of an aerosol delivery system in accordance with some embodiments of the disclosure.

FIG. 2 is a schematic perspective view of an aerosol delivery system in accordance with some embodiments of the disclosure, comprising a baffle assembly for adjusting air flow through the system.

FIG. 3 is a schematic perspective end view of an aerosol delivery subsystem comprising a baffle assembly for adjusting air flow, in accordance with some embodiments of the disclosure.

FIGS. 4a-c are schematic perspective views of an aerosol delivery subsystem in accordance with a first particular embodiment of the disclosure.

FIGS. 5a-e are schematic perspective views of an aerosol delivery subsystem in accordance with a second particular embodiment of the disclosure.

FIGS. 6a-c are schematic perspective views of an aerosol delivery subsystem in accordance with a third particular embodiment of the disclosure.

FIGS. 7a-c are schematic perspective views of an aerosol delivery subsystem in accordance with a fourth particular embodiment of the disclosure.

FIGS. 8a-c are schematic illustrations of how the baffle assembly of the first particular embodiment of FIGS. 4a-c moves to adjust air flow through the subsystem.

FIGS. 9a-c are schematic illustrations of how the baffle assembly of the second particular embodiment of FIGS. 5a-d moves to adjust air flow through the subsystem.

FIGS. 10a-c are schematic illustrations of how the baffle assembly of the third particular embodiment of FIGS. 6a-c moves to adjust air flow through the subsystem.

FIGS. 11a-c are schematic illustrations of how the baffle assembly of the fourth particular embodiment of FIGS. 7a-c moves to adjust air flow through the subsystem.

FIGS. 12a-c are schematic views of an aerosol delivery subsystem in accordance with a fifth particular embodiment of the disclosure.

### DETAILED DESCRIPTION OF THE DISCLOSURE

Aspects and features of certain examples and embodiments are described herein. Some aspects and features of certain examples and embodiments may be implemented conventionally and these are not described in detail in the interest of brevity. It will thus be appreciated that aspects and features of apparatuses and methods discussed herein which are not described in detail may be implemented in accordance with any suitable conventional techniques.

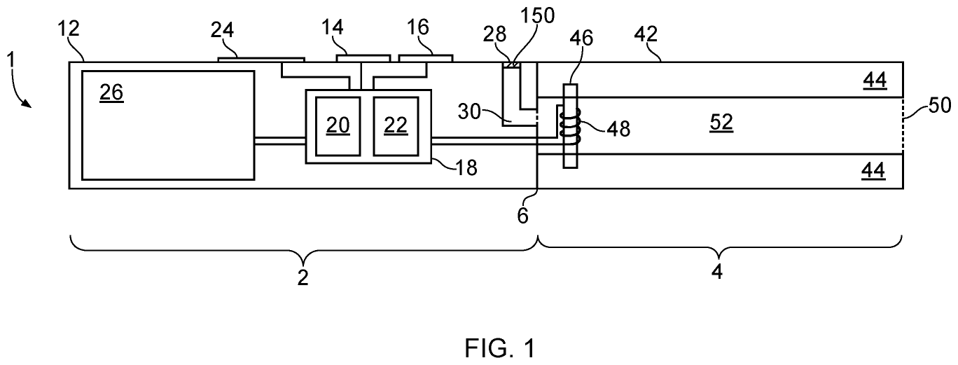

FIG. 1 is a cross-sectional view through an example aerosol delivery system 1 in accordance with certain embodiments of the disclosure, providing an introduction to two-part aerosol delivery systems, the components therein and their functionality. The system 1 comprises a baffle assembly 150, which is described in more detail later, with reference to the subsequent figures.

In this first embodiment, the aerosol delivery system 1 comprises two main parts, namely a reusable part 2 and a replaceable/disposable consumable cartridge part 4. In normal use, the reusable part 2 and the cartridge part 4 are releasably coupled together at an interface 6. When the cartridge part 4 is exhausted or the user simply wishes to switch to a different cartridge part 4, the cartridge part 4 may be removed from the reusable part 2 and a replacement cartridge part 4 attached to the reusable part 2 in its place. The interface 6 provides a structural, electrical and airflow path connection between the two parts 2, 4 and may be established in accordance with conventional techniques, for example based around a screw thread, magnetic or bayonet fixing with appropriately arranged electrical contacts and openings for establishing the electrical connection and airflow path between the two parts 2, 4 as appropriate. The specific manner by which the cartridge part 4 mechanically mounts to the reusable part 2 is not significant to the principles described herein, but for the sake of a concrete example is assumed here to comprise a magnetic coupling (not represented in FIG. 1). It will also be appreciated the interface 6 in some implementations may not support an electrical and/or airflow path connection between the respective parts 2, 4. For example, in some implementations an aerosol generator may be provided in the reusable part 2 rather than in the cartridge part 4, or the transfer of electrical power from the reusable part 2 to the cartridge part 4 may be wireless (e.g. based on electromagnetic induction), so that an electrical connection between the reusable part 2 and the cartridge part 4 is not needed. Furthermore, in some implementations the airflow through the electronic cigarette might not go through the reusable part 2, so that an airflow path connection between the reusable part 2 and the cartridge part 4 is not needed. In some instances, a portion of the airflow path may be defined at the interface between portions of the reusable part 2 and cartridge part 4 when these are coupled together for use.

The cartridge/consumable part 4 may in accordance with certain embodiments of the disclosure be broadly conventional. In FIG. 1, the cartridge part 4 comprises a cartridge housing 42 formed of a plastics material. The cartridge housing 42 supports other components of the cartridge part 4 and provides the mechanical interface 6 with the reusable part 2. The cartridge housing 42 is generally circularly symmetric about a longitudinal axis along which the cartridge part 4 couples to the reusable part 2. In this example, the cartridge part 4 has a length of around 4 cm and a diameter of around 1.5 cm. However, it will be appreciated the specific geometry, and more generally the overall shapes and materials used, may be different in different implementations.

Within the cartridge housing 42 is a chamber or reservoir 44 that contains aerosol-generating material. In the example shown schematically in FIG. 1, the reservoir 44 stores a supply of liquid aerosol generating material. In this example, the liquid reservoir 44 has an annular shape with an outer wall defined by the cartridge housing 42 and an inner wall that defines an airflow path 52 through the cartridge part 4. The reservoir 44 is closed at each end with end walls to contain the aerosol generating material. The reservoir 44 may be formed in accordance with conventional techniques, for example it may comprise a plastics material and be integrally moulded with the cartridge housing 42.

The cartridge/consumable part 4 further comprises an aerosol generator 48 located towards an end of the reservoir 44 opposite to a mouthpiece outlet 50. It will be appreciated that in a two-part system such as shown in FIG. 1, the aerosol generator 48 may be in either of the reusable part 2 or the cartridge part 4. For example, in some embodiments, the aerosol generator 48 (e.g. a heater, which may be in the form of a wick and coil arrangement as shown, a ceramic heater, a distiller, which may be formed from a sintered metal fibre material or other porous conducting material, or any suitable alternative aerosol generator) may be comprised in the reusable part 2, and is brought into proximity with a portion of aerosol generating material in the cartridge part 4 when the cartridge part 4 is engaged with the reusable part 2. In such embodiments, the cartridge part 4 may comprise a portion of aerosol generating material, and an aerosol generator 48 comprising a heater is at least partially inserted into or at least partially surrounds the portion of aerosol generating material as the cartridge part 4 is engaged with the reusable part 2.

In the example of FIG. 1, a wick 46 in contact with the aerosol generator 48 extends transversely across the cartridge airflow path 52 with its ends extending into the reservoir 44 of the liquid aerosol generating material through openings in the inner wall of the reservoir 44. The openings in the inner wall of the reservoir 44 are sized to broadly match the dimensions of the wick 46 to provide a reasonable seal against leakage from the liquid reservoir 44 into the cartridge airflow path without unduly compressing the wick 46, which may be detrimental to its fluid transfer performance.

The wick 46 and aerosol generator 48 are arranged in the cartridge airflow path 52 such that a region of the cartridge airflow path 52 around the wick 46 and heater 48 in effect defines a vaporisation region for the cartridge part 4. Aerosol generating material in the reservoir 44 infiltrates the wick 46 through the ends of the wick extending into the reservoir 44 and is drawn along the wick by surface tension/capillary action (i.e. wicking). The aerosol generator 48 in this example comprises an electrically resistive wire coiled around the wick 46. In the example of FIG. 1, the heater 48 comprises a nickel chrome alloy (Cr20Ni80) wire and the wick 46 comprises a glass fibre bundle, but it will be appreciated the specific aerosol generator configuration is not significant to the principles described herein. In use, electrical power may be supplied to the aerosol generator 48 to vaporise an amount of aerosol generating material (aerosol generating material) drawn to the vicinity of the aerosol generator 48 by the wick 46. Vaporised aerosol generating material may then become entrained in air drawn along the cartridge airflow path from the vaporisation region towards the mouthpiece outlet 50 for user inhalation.

As noted above, the rate at which aerosol generating material is vaporised by the aerosol generator 48 will depend on the amount (level) of power supplied to the aerosol generator 48. Thus electrical power can be applied to the aerosol generator 48 to selectively generate aerosol from the aerosol generating material in the cartridge part 4, and furthermore, the rate of aerosol generation can be changed by changing the amount of power supplied to the aerosol generator 48, for example through pulse width and/or frequency modulation techniques.

The reusable part 2 comprises a housing 12 having with an opening that defines an air inlet 28 for the e-cigarette, a power source 26 (for example a battery) for providing operating power for the electronic cigarette, control circuitry/controller 22 for controlling and monitoring the operation of the electronic cigarette, a first user input button 14, a second user input button 16, and a visual display 24. The reusable part 2 also comprises a baffle assembly 150 across the inlet 28. The baffle assembly 150 is adjustable to alter air flow through the downstream air inlet 28 and is described in more detail later, with reference to the subsequent figures.

The housing 12 may be formed, for example, from a plastics or metallic material and in this example has a circular cross section generally conforming to the shape and size of the cartridge part 4 so as to provide a smooth transition between the two parts 2, 4 at the interface 6. In this example, the reusable part 2 has a length of around 8 cm so the overall length of the e-cigarette when the cartridge part 4 and the reusable part 2 are coupled together is around 12 cm. However, and as already noted, it will be appreciated that the overall shape and scale of an electronic cigarette implementing an embodiment of the disclosure is not significant to the principles described herein.

The air inlet 28 connects to an airflow path 51 through the reusable part 2. The airflow path 51 in turn connects to the cartridge airflow path 52 across the interface 6 when the reusable part 2 and cartridge part 4 are connected together. Thus, when a user inhales on the mouthpiece opening 50, air is drawn in through the air inlet 28, along the reusable part airflow path 51, across the interface 6, through the aerosol generation area in the vicinity of the aerosol generator 48 (where vaporised aerosol generating material becomes entrained in the air flow), along the cartridge airflow path 52, and out through the mouthpiece opening 50 for user inhalation.

The power source 26 in this example is rechargeable and may be of a conventional type, for example of the kind normally used in electronic cigarettes and other applications requiring provision of relatively high currents over relatively short periods. The power source 26 may be recharged through a charging connector in the reusable part housing 12, for example a USB connector.

First and/or second user input buttons 14, 16 may be provided, which in this example are conventional mechanical buttons, for example comprising a spring mounted component which may be pressed by a user to establish an electrical contact. In this regard, the input buttons may be considered input devices for detecting user input and the specific manner in which the buttons are implemented is not significant. The buttons may be assigned to functions such as switching the aerosol delivery system 1 on and off, and adjusting user settings such as a power to be supplied from the power source 26 to the aerosol generator 48. However, the inclusion of user input buttons is optional, and in some embodiments buttons may not be included.

A display 24 may be provided to give a user with a visual indication of various characteristics associated with the aerosol delivery system, for example current power setting information, remaining power source power, and so forth. The display may be implemented in various ways. In this example the display 24 comprises a conventional pixilated LCD screen that may be driven to display the desired information in accordance with conventional techniques. In other implementations, the display may comprise one or more discrete indicators, for example LEDs, that are arranged to display the desired information, for example through particular colours and/or flash sequences. More generally, the manner in which the display 24 is provided and information is displayed to a user using the display is not significant to the principles described herein. For example, some embodiments may not include a visual display and/or may include other means for providing a user with information relating to operating characteristics of the aerosol delivery system, for example using audio signalling, or may not include any means for providing a user with information relating to operating characteristics of the aerosol delivery system.

A controller 22 is suitably configured/programmed to control the operation of the aerosol delivery system 1 to provide functionality in accordance with embodiments of the disclosure as described further herein, as well as for providing conventional operating functions of the aerosol delivery system 1 in line with the established techniques for controlling such devices. The controller (processor circuitry) 22 may be considered to logically comprise various sub-units/circuitry elements associated with different aspects of the operation of the aerosol delivery system 1. In this example the controller 22 comprises power supply control circuitry for controlling the supply of power from the power source 26 to the aerosol generator 48 in response to user input, user programming circuitry 20 for establishing configuration settings (e.g. user-defined power settings) in response to user input, as well as other functional units/circuitry associated functionality in accordance with the principles described herein and conventional operating aspects of electronic cigarettes, such as display driving circuitry and user input detection circuitry. It will be appreciated that the functionality of the controller 22 can be provided in various different ways, for example using one or more suitably programmed programmable computer(s) and/or one or more suitably configured application-specific integrated circuit(s)/circuitry/chip(s)/chipset(s) configured to provide the desired functionality.

The functionality of the controller 22 is described further herein. For example, the controller 22 may comprise an application specific integrated circuit (ASIC) or microcontroller, for controlling the aerosol delivery device. The microcontroller or ASIC may include a CPU or micro-processor. The operations of a CPU and other electronic components are generally controlled at least in part by software programs running on the CPU (or other component). Such software programs may be stored in non-volatile memory, such as ROM, which can be integrated into the microcontroller itself, or provided as a separate component. The CPU may access the ROM to load and execute individual software programs as and when required.

The reusable part 2 comprises an airflow sensor 30 which is electrically connected to the controller 22. In most embodiments, the airflow sensor 30 comprises a so-called “puff sensor”, in that the airflow sensor 30 is used to detect when a user is puffing on the device. In some embodiments, the airflow sensor 30 comprises a switch in an electrical path providing electrical power from the power source 26 to the aerosol generator 48. In such embodiments, the airflow sensor 30 generally comprises a pressure sensor configured to close the switch when subjected to a particular range of pressures, enabling current to flow from the power source 26 to the aerosol generator 48 once the pressure in the vicinity of the airflow sensor 30 drops below a threshold value. The threshold value can be set to a value determined by experimentation to correspond to a characteristic value associated with the initiation of a user puff. In other embodiments, the airflow sensor 30 is connected to the controller 22, and the controller distributes electrical power from the power source 26 to the aerosol generator 48 in dependence of a signal received from the airflow sensor 30 by the controller 22. The specific manner in which the signal output from the airflow sensor 30 (which may comprise a measure of capacitance, resistance or other characteristic of the airflow sensor, made by the controller 22) is used by the controller 22 to control the supply of power from the power source 26 to the aerosol generator 48 can be carried out in accordance with any approach known to the skilled person.

In the example shown in FIG. 1, the airflow sensor 30 is mounted to a printed circuit board (PCB) 31, but this is not essential. The airflow sensor 30 may comprise any sensor which is configured to determine a characteristic of airflow in an airflow path 51 disposed between air inlet 28 and mouthpiece opening 50, for example a pressure sensor or transducer (for example a membrane or solid-state pressure sensor), a combined temperature and pressure sensor, or a microphone (for example an electret-type microphone), which is sensitive to changes in air pressure, including acoustical signals. The airflow sensor 30 is situated within a sensor cavity or chamber 32, which comprises the interior space defined by one or more chamber walls 34. The sensor cavity 32 comprises a region internal to one or more chamber walls 34 in which an airflow sensor 30 can be fully or partially situated. In some embodiments, the PCB 31 comprises one of the chamber walls of a sensor housing comprising the sensor chamber/cavity 32.

A deformable membrane is disposed across an opening communicating between the sensor cavity 32 containing the sensor 30, and a portion of the airflow path disposed between air inlet 28 and mouthpiece opening 50. The deformable membrane covers the opening, and is attached to one or more of the chamber walls according to approaches described further herein.

As described further herein, the aerosol delivery system 1 comprises communication circuitry configured to enable a connection to be established with one or more further electronic devices (for example, a storage/charging case, and/or a refill/charging dock) to enable data transfer between the aerosol delivery system 1 and further electronic device(s). In some embodiments, the communication circuitry is integrated into controller 22, and in other embodiments it is implemented separately (comprising, for example, separate application-specific integrated circuit(s)/circuitry/chip(s)/chipset(s)). For example, the communication circuitry may comprise a separate module to the controller 22 which, while connected to controller 22, provides dedicated data transfer functionality for the aerosol delivery device. In some embodiments, the communication circuitry is configured to support communication between the aerosol delivery system 1 and one or more further electronic devices over a wireless interface. The communication circuitry may be configured to support wireless communications between the aerosol delivery system 1 and other electronic devices such as a case, a dock, a computing device such as a smartphone or PC, a base station supporting cellular communications, a relay node providing an onward connection to a base station, a wearable device, or any other portable or fixed device which supports wireless communications.

Wireless communications between the aerosol delivery system 1 and a further electronic device may be configured according to data transfer protocols such as Bluetooth®, ZigBee, WiFi®, Wifi Direct, GSM, 2G, 3G, 4G, 5G, LTE, NFC, RFID, or generally any other wireless, and/or wired, network protocol or interface. The communication circuitry may comprise any suitable interface for wired data connection, such as USB-C, micro-USB or Thunderbolt interfaces, and may comprise pin or contact pad arrangements configured to engage cooperating pins or contact pads on a dock, case, cable, or other external device which can be connected to the aerosol delivery system 1.

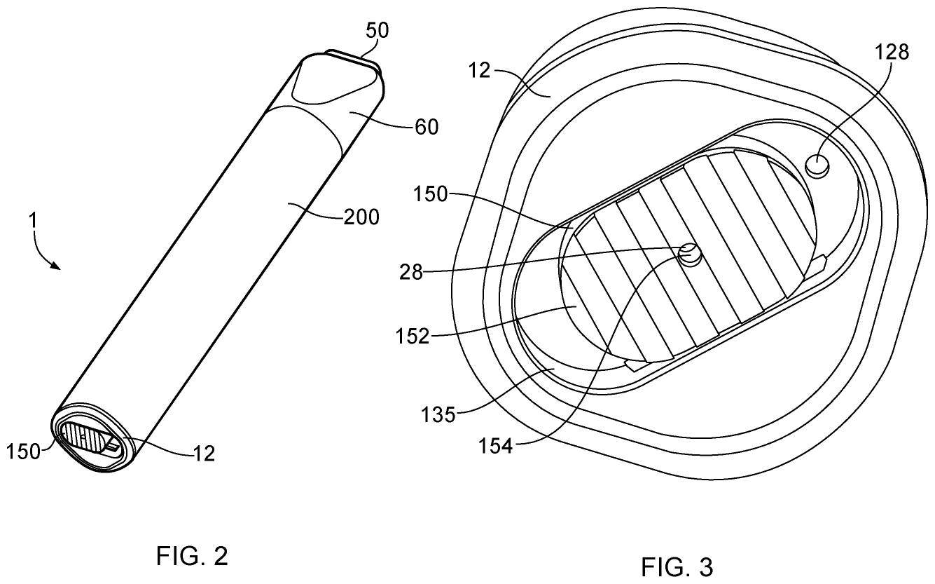

FIG. 2 is a schematic perspective view of an aerosol delivery system 1 in accordance with some embodiments of the disclosure, comprising a baffle assembly 150 for a user to adjust air flow through the system 1. In contrast to FIG. 1, the baffle assembly 150 is located at a proximal end of the system 1, rather than at the interface 6 between the device and cartridge parts 2,4. The system 1 of FIG. 2, and any other embodiment, may comprise a two-part reusable system as in FIG. 1, or be a single-use disposable unitary or two-part system. The system 1 also comprises a mouthpiece housing 60 at distal end thereof, leading to the mouthpiece outlet 50.

FIG. 3 is a schematic perspective end view of an aerosol delivery subsystem 100 comprising a baffle assembly 150 for adjusting air flow. The subsystem 100 may generally form part of an aerosol delivery system 1 and in particular may form part of the reusable device 2 and/or the consumable cartridge 4. The baffle assembly 150 advantageously enables a user to alter the flow of air into the system 1, and thereby alter the pressure drop experienced as they inhale on the mouthpiece. This provides adjustable operating characteristics to target certain characteristics which may be desirable to a user.

The variable air flow can be used to adjust the draw resistance of the aerosol delivery system. As a user inhales, the lungs in effect work against the draw resistance, i.e. the work required to pull air into and then through the system into the lungs. For most users, there is a range of draw resistance that helps them to perform a steady inhalation. However, if the draw resistance is too low, the inhalation may become too rapid and unsteady, while if the draw resistance is too high, the inhalation may become unduly burdensome. The most suitable level of draw resistance varies from one user to another user, based e.g. on physiological factors. Accordingly, providing variable ventilation and fluid (air) flow volume as described herein can help a user to configure the draw resistance to an appropriate value for their own personal preferences and characteristics.

In the example of FIG. 3, the subsystem 100 comprises a housing 12 having openings that define air inlets 28, 128, and a baffle assembly cavity 135 for receiving the baffle assembly 150. The baffle assembly 150 comprises a baffle slider 152 and has an aperture 154 therethrough. In FIG. 3, the baffle aperture 154 is aligned with the first opening in a centre of the housing 12 defining the first air inlet 28, providing an air flow path through both the baffle assembly 150 and the housing 12 when the first air inlet 28 and the baffle aperture 154 are aligned, as shown. The housing 12 also has a second opening offset from the centre, which is not obscured by the baffle assembly 150, forming a second (bypass) air inlet 128.

The baffle assembly 150 is movable with respect to the first air inlet 28 in the housing 12 and the subsystem 100 further comprises an engagement mechanism for positively engaging the baffle assembly 150 with respect to the subsystem 100 in at least one of a plurality of predetermined positions as the baffle assembly 150 is moved with respect to the air inlet 28 (or equally, with respect to the subsystem 100 or any static part thereof such as the housing 12), wherein different ones of the plurality of predetermined positions (e.g. a fully open position, a fully closed position and at least one discrete intermediate position) allow different amounts of air flow into the system, e.g. different positions result in different degrees of alignment between the baffle assembly aperture 154 and the air inlet 28, to alter air flow through the air inlet 28.

In some embodiments, the engagement mechanism comprises one or more cooperating, contacting, engaging or interlocking pairs of protrusions and recesses. Any suitable shape, structure and combination of protrusions and recesses may be used, such as a spherical cap, hemisphere or interlocking shape or other detent mechanism. In particular, in some embodiments, the engagement mechanism comprises the baffle assembly 150 having a baffle assembly protrusion 157 or a recess 158 and the subsystem 100 (i.e. any other subcomponent thereof) having a complementary protrusion 257 or recess 258, for engaging with the protrusion 157 or recess 158 on the baffle assembly 150. Moreover, in some embodiments, the engagement mechanism comprises a baffle assembly ramp 159 and/or a complementary ramp 259 for guiding and/or biasing the baffle assembly 150 into one or more of the plurality of predetermined positions.

In some embodiments, the baffle assembly 150 comprises one or more snap-fit projections 155 for securing the baffle assembly 150 within the subsystem 100. Such snap-fit projections 155 are beneficial because they provide a secure tool-less construction. In some embodiments, the baffle snap-fit projection 155 comprises one or more cantilever, torsional and/or annular snap-fit projections 155; the baffle assembly protrusion 157 or recess 158; and/or the baffle assembly ramp 159.

In some embodiments, the baffle assembly 150 comprises a baffle assembly seal 156, for sealing around the baffle assembly 150 or a part thereof, such as the slider 152, to minimise leakage of fluid in use (particularly liquid from a cartridge having liquid in a reservoir), and/or leakage of air into or out of the baffle assembly 150 or wider system 1. In some embodiments, the seal 156 comprises the baffle assembly protrusion 157 or recess 158. The seal 156 is compressible and may comprise silicone.

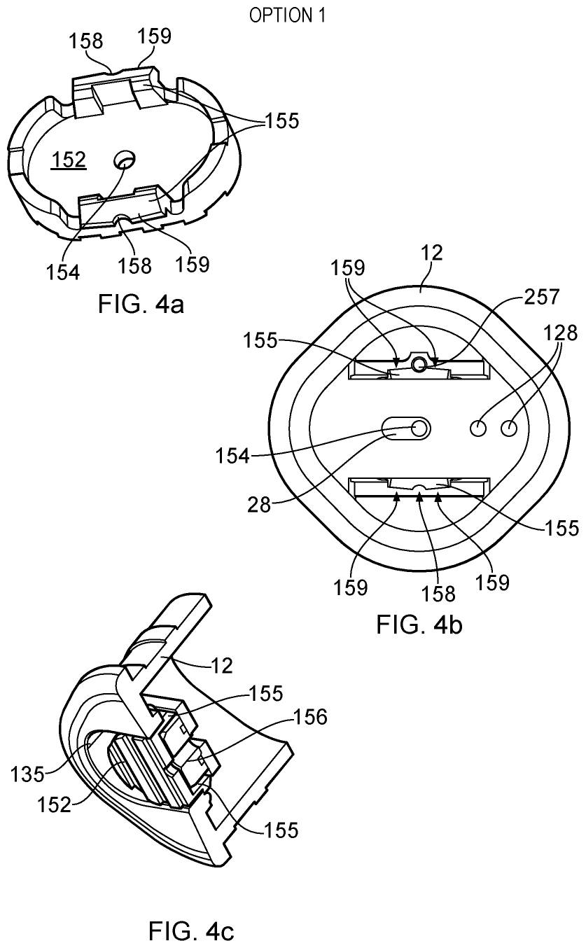

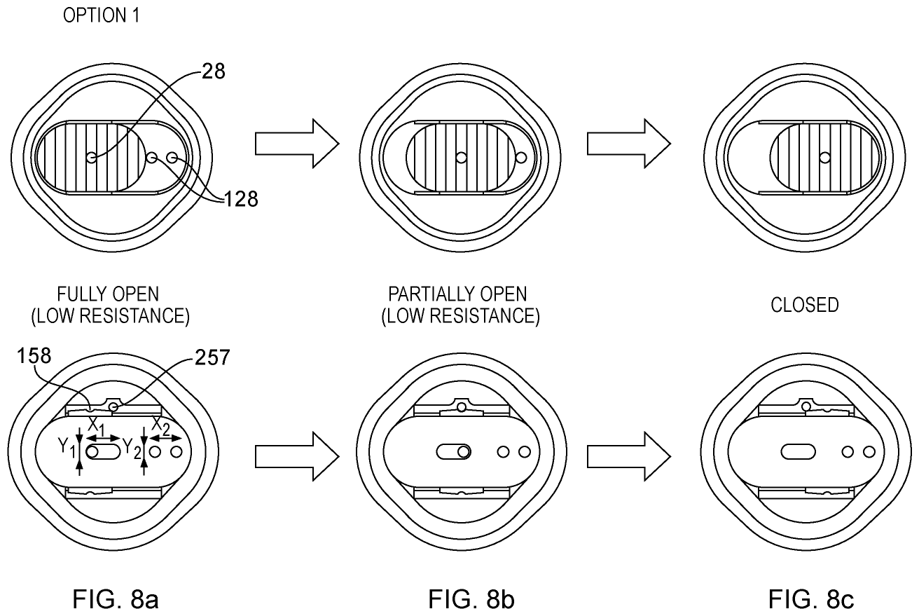

FIGS. 4-11 illustrate particular example embodiments of the disclosure. All embodiments are provided as a representative examples only and are not exhaustive and/or exclusive. The features of these embodiments are contemplated in isolation and any features may selectively be combined in any combination. In essence, these embodiments detail mechanical engagement or interlocking arrangements to provide a high friction path between low friction end points which correspond to each engagement position (air flow configuration), to give feedback to the user so they can understand which position they are in. The plurality of predetermined positions generally include fully open, fully closed and one or more distinct partially open (intermediate) configurations, and these are discussed in more detail later with reference to FIGS. 8-11. Preferably, the subsystem comprises the engagement mechanism for providing positive engagement in at least one partially open configuration, i.e. at least one intermediate position between the fully open and fully closed positions, to confirm to a user that they are in a predetermined, intermediate (e.g. 25%, 50%, 75% of inlets are open) position. In some embodiments, the fully open and fully closed configurations may correspond to end points for the baffle assembly 150 and thus these configurations do not necessarily require a positive engagement mechanism to inform the user that they are in that configuration, although this may still be beneficial, e.g. to provide a child safety lock feature in the closed configuration.

FIGS. 4a-c are schematic views of an aerosol delivery subsystem 100 in accordance with a first particular embodiment of the disclosure. FIG. 4a illustrates the slider 152 of the baffle assembly 150. Here, the slider 152 is sized and shaped to be operable by a user's fingers or thumb from a base of the system (see FIG. 4c)—the slider 152 is discorectangular in shape (a stadium) and has a length of about 10 mm and a width of about 5 mm. More generally, the slider may be about 5-50 mm long and 2-25 mm wide.

The slider 152 comprises the aperture 154 therethrough and two snap-fit projections 155 in the form of cantilever projections on opposing long sides of the slider 152, for securing the baffle assembly 150 to the housing 12. In FIG. 4a, the snap-fit projections 155 each comprise the baffle assembly recess 158 in the form of a hemispherical groove or spherical cap recess 158, providing secure engagement and user feedback confirming the same. The snap-fit projections 155 also comprise a tapering wall forming a baffle assembly ramp 159, best shown in FIG. 4b, for guiding and/or biasing the baffle assembly 150 between and into the predetermined positions, enhancing the tactile feedback to the user as they engage the mechanism to alter air flow through the system 1. FIGS. 8a-c, discussed later, illustrate how the baffle assembly 150 moves between the predetermined positions for this first particular embodiment.

FIG. 4b also shows that the housing 12 comprises a complementary protrusion 257 in the form of a spherical cap 257 for positively engaging with the baffle assembly recess 158. Only one cap 257 is shown in FIG. 4b for clarity, although in embodiments, any number and/or arrangement of protrusions 157, 257 and recesses 158, 258 may be used-in particular a second, opposing complementary protrusion 257 is contemplated for engaging the opposing baffle assembly recess 158. FIG. 4b also shows four baffle assembly ramps 159 on the snap-fit projections 155, one each side of the recess 158. Further, the housing 12 has 3 openings: one elongate oval opening defining a central air inlet 28 as well as two side bypass inlets 128. In this arrangement, the baffle assembly 150 is movable to cover or align the aperture 154 with the air inlet 28.

The subsystem may comprise a fluid flow sensor 30 for detecting air flow through the air inlet 28, in some embodiments with a sensor seal 130 having a cavity for receiving/locating the sensor 30 in the subsystem and providing an air flow path thereto. Preferably, the fluid flow sensor 30 is located proximal or adjacent to at least one of the air inlets 28 and/or a sensing portion of the fluid flow sensor 30 is directly exposed to or within a fluid flow path from the air inlet 28 towards an aerosol generator 48. The seal 130 is compressible and may comprise silicone.

The bypass inlets 128 provide secondary inlets to the system that bypass some components of the subsystem, for example bypassing the baffle assembly 150, the aperture 154 therethrough and/or the fluid flow sensor 30. The bypass inlets 128 may still be covered by the baffle assembly 150 in one or more of the predetermined positions to prevent air flow therethrough. The primary air inlets 28 and the bypass air inlets 128 may all be the same size, e.g. approximately 0.5-5.0 mm, 0.5-3.0 mm, 0.5-2.5 mm or substantially 1.0-2.0 mm each in diameter, or they may be of different sizes or shapes. In some embodiments, the outermost bypass air inlet 128 is larger in diameter than the innermost bypass air inlet 128. Varying the number and/or size of the air inlets 28, 128 allows fine tuning of the pressure drop performance of the device.

In some embodiments, the subsystem comprises a fluid flow sensor 30 and multiple air inlets 28, 128, wherein the multiple air inlets 28, 128 comprise one or more primary air inlets 28 to the fluid flow sensor 30 and one or more secondary, bypass air inlets 128 bypassing the fluid flow sensor 30, optionally wherein:
a. the primary air inlets 28 to the fluid flow sensor 30 are configured to deliver air to an aerosol generator 48 in use; and/or
b. the secondary, bypass air inlets 128 are configured to deliver air to an aerosol generator 48 in use; and/or
c. the secondary, bypass air inlets 128 are configured to provide airflow through the system, bypassing the aerosol generator 48 in use.

In some embodiments, the subsystem is configured to:
enable a supply of power to an aerosol generator 48 when one or more of the primary air inlets 28 are open and provide an air flow path therethrough; and/or
enable a supply of power to an aerosol generator 48 when one or more of the primary or secondary air inlets 28, 128 are open and provide an air flow path therethrough; and/or
adjust a supply of power to an aerosol generator 48 when moving between predetermined positions providing non-zero degrees of alignment between the baffle assembly aperture 154 and the primary air inlets 28; and/or
disable a supply of power to an aerosol generator 48 when all primary air inlets 28 are closed; and/or
disable a supply of power to an aerosol generator 48 when all air inlets 28, 128 are closed, for example to provide a child safety lock.

The subsystem 100 may be configured to activate the aerosol generator 48 in response to the fluid flow sensor 30 sensing fluid (air) flow and/or disable the aerosol generator 48 when no fluid (air) flow is sensed. The fluid flow sensor 30 or the sensor seal 130 may comprise the complementary protrusion 257 or recess 258 for engaging with the baffle assembly protrusion 157 or recess 258.

FIG. 4c shows an axial, vertical cross-section through the subsystem 100 and shows that the baffle assembly 150 comprises a baffle assembly seal 156, for sealing around the baffle assembly aperture 154. FIG. 4c also shows that the baffle assembly slider 152 has surface features in the form of a plurality of alternating ridges and recesses extending along its length, the ridges and recesses extending perpendicular to an axis of movement of the baffle between the predetermined positions, providing a tactile feel to the slider 152. In further embodiments, the baffle assembly slider 152 comprises one or more curved recesses, e.g. a fillet, for receiving part of a user's finger or thumb, to engage the slider 152 in use. In one embodiment, the slider 152 comprises a curved recess at each end of its length and/or a central curved recess. In other embodiments, other surface features such as knurling may be provided. Such surface features may provide a more comfortable grip and make the slider 152 easier to operate.

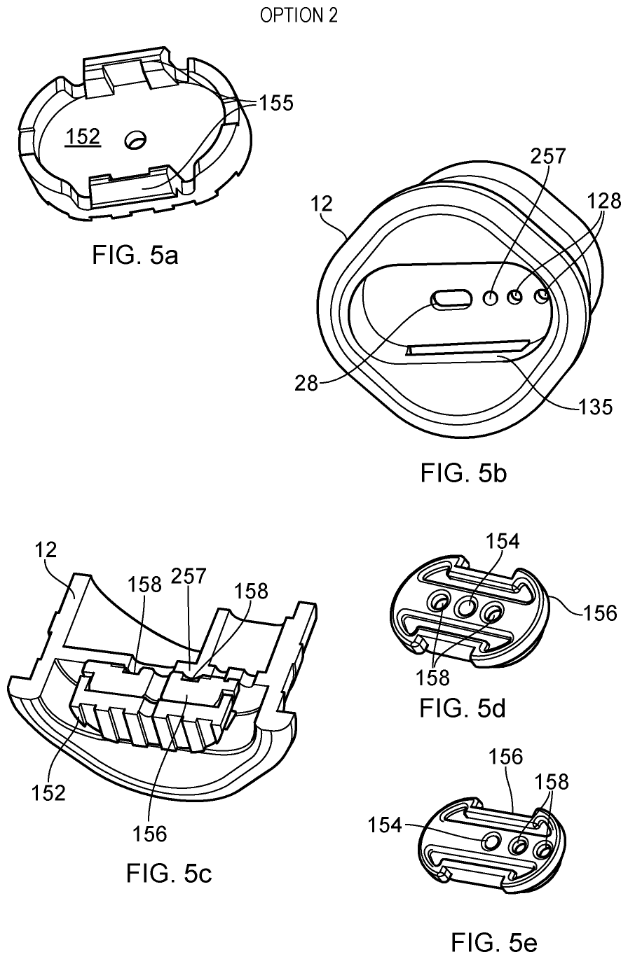

FIGS. 5a-d are schematic views of an aerosol delivery subsystem in accordance with a second particular embodiment of the disclosure. Key differences to the first particular embodiment of FIGS. 4a-c are described herein. In particular, in this embodiment, whilst the slider 152 comprises the baffle assembly aperture 154 therethrough and snap-fit projections 155 in the form of cantilever projections on opposing long sides of the slider 152, the slider 152 does not comprise the baffle assembly protrusion 157 or recess 158, nor any ramp 159. Instead, the baffle assembly seal 156 between the slider 152 and the housing 12 (see FIGS. 5c-d) comprises an aperture therethrough (continuing the baffle assembly aperture 154 in one or more open positions), and the baffle assembly seal 156 comprises two recesses 158 in the form of hemispherical grooves 158, one either side of the aperture 154.

As shown in FIGS. 5b-c, the housing 12 comprises a complementary protrusion 257 in the form of a spherical cap 257 between the elongate primary air inlet 28 and two secondary, bypass inlets 128, for engaging with one or more of the baffle assembly recesses 158.

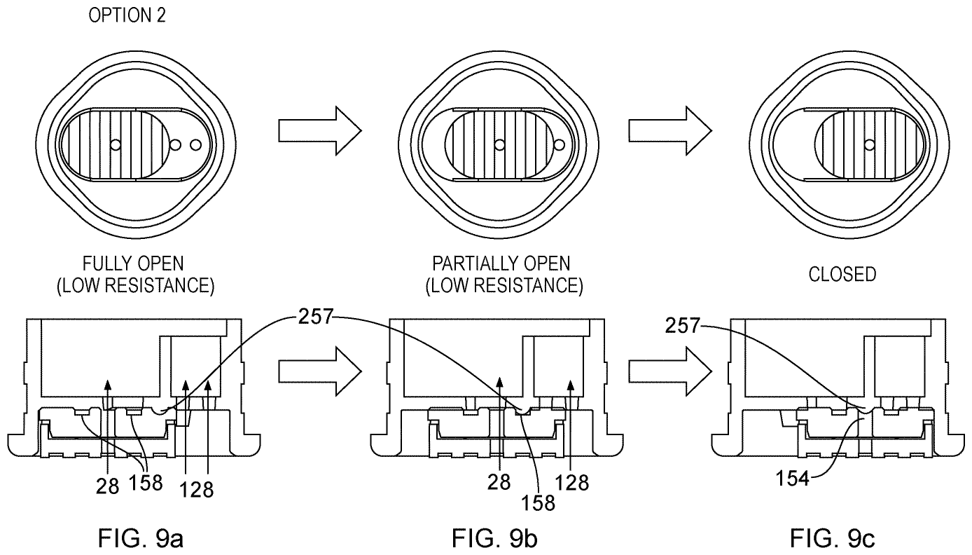

FIG. 5c is a vertical cross-section showing the subsystem 100 in a partially-open configuration (see FIG. 9b, discussed later), with the protrusion 257 engaged with the right-hand one of the baffle assembly recesses 158. As outlined above, the complementary protrusion 257 engages with the right-hand baffle assembly recess 158 as the slider moves into the partially open configuration of FIG. 5c (also shown in FIG. 9a, discussed later), providing a predetermined partially-open configuration, and the complementary protrusion 257 engages the baffle assembly aperture 154 when the slider 152 moves to the right, into a closed configuration (see FIG. 9c, discussed later).

FIG. 5d shows the baffle seal 156 in isolation, illustrating the central aperture 154 between left and right baffle assembly recesses 158. Although FIG. 5d depicts one recess 158 on either side of the aperture 154, the left-hand recess 158 may not necessarily be used to engage with a protrusion 257 and instead may be provided simply to aid assembly, so that the seal 156 is symmetrical and may thus be installed either way round.

FIG. 5e shows a variant of the baffle seal 156 of FIG. 5d, having two recesses 158 to the right of the central aperture 154. The configuration of FIG. 5e does not have the symmetrical nature of the FIG. 5d variant, but the two (right) recesses 158 provides positive engagement feedback as the baffle assembly 150 moves into each of the predetermined open, partially open and closed configurations, i.e. additionally providing feedback when moving into the fully open position, in contrast to the baffle seal 156 of FIG. 5d. FIGS. 9a-c illustrate movement of the baffle seal 156 of FIG. 5d into each position/configuration.

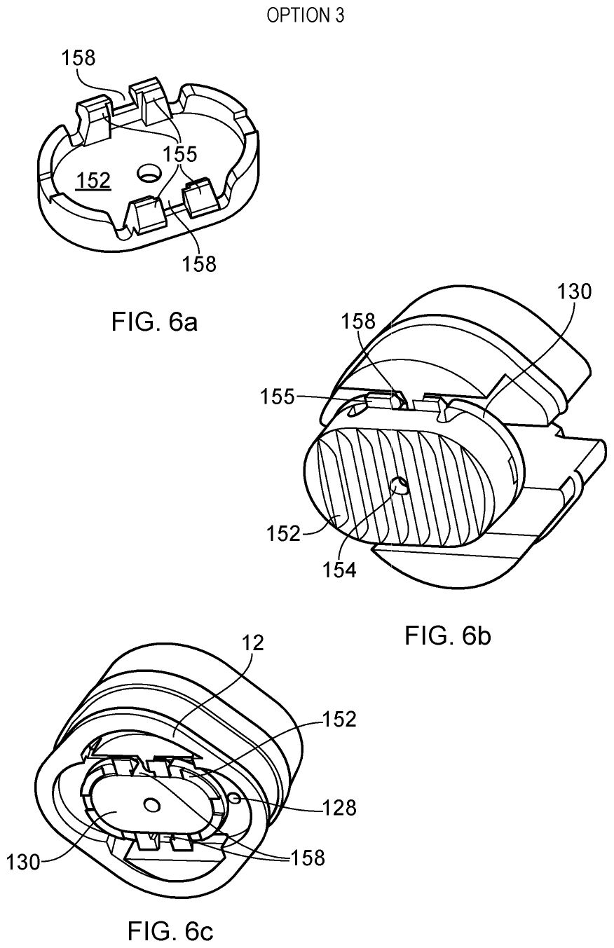

FIGS. 6a-c are schematic views of an aerosol delivery subsystem in accordance with a third particular embodiment of the disclosure. Key differences to the first particular embodiment of FIGS. 4a-c are described herein. FIG. 6a illustrates the slider 152 of the baffle assembly 150. Here, the slider 152 comprises the aperture 154 therethrough, whilst instead of two elongate projections 155 with recesses 158 and ramps 159 as in FIG. 4a, in FIG. 6a, each side of the slider 152 comprises a pair of snap-fit cantilever projections 155, each pair forming a baffle assembly recess 158 therebetween.

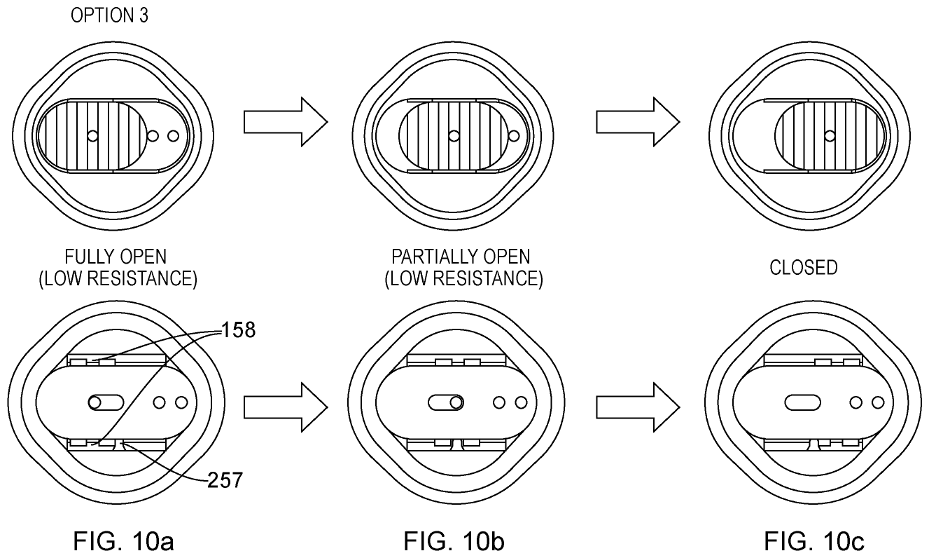

FIGS. 6b-c illustrate that the subsystem 100 comprises a sensor seal 130 for housing the air flow sensor 30 proximal or adjacent to the inlet 28, to detect air flow therethrough. FIG. 6c is a cross-section view through the slider 152, showing the baffle assembly recesses 158 on the slider 152, where the seal 130 comprises complementary protrusions 257 on opposing sides thereof (not shown), for positively engaging with the baffle assembly recesses 158 on the baffle slider 152. (The protrusions 257 on the seal 130 are not shown in FIG. 6c as the cross-section is through the middle of the seal 130 and not through the engaging/sealing surface-see FIGS. 10a-10c showing the protrusions 257 on the seal 130).

As shown in FIG. 6c, in this embodiment, the secondary, bypass inlets 128 bypass the air flow sensor 30, whilst the central primary air inlet 28 provides air flow to the sensor 30 (not shown, but housed within the seal 130), which can be used to trigger activation/deactivation of the device, e.g. providing/preventing the supply of power to an aerosol generator 48.

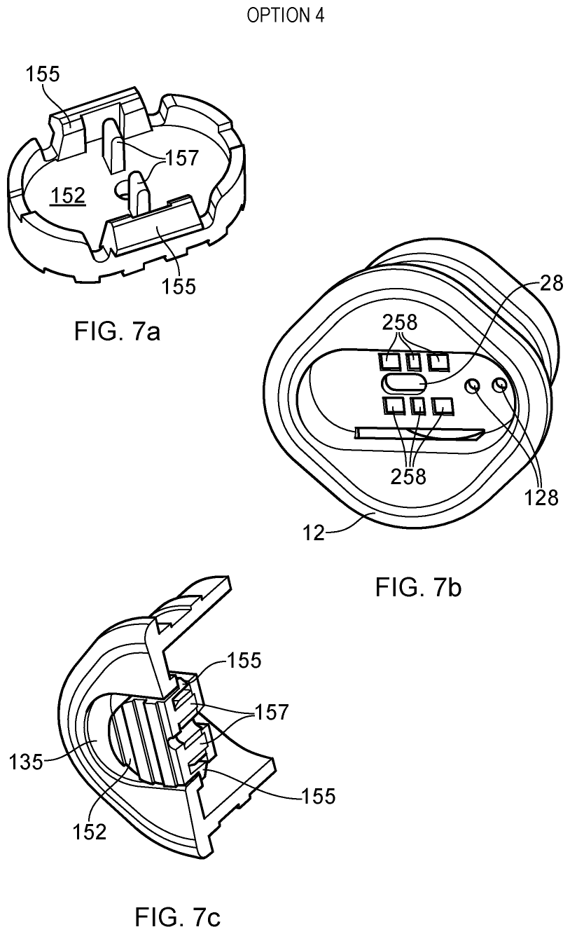

FIGS. 7a-c are schematic views of an aerosol delivery subsystem in accordance with a fourth particular embodiment of the disclosure. Key differences to the first particular embodiment of FIGS. 4a-c are described herein. In particular, in this embodiment, whilst the slider 152 comprises the baffle assembly aperture 154 therethrough and snap-fit projections 155 in the form of cantilever projections on opposing long sides of the slider 152, the slider 152 comprises cantilever baffle assembly protrusions 157 instead of the recesses 158 and ramps 159 of FIG. 4a. The housing 12 comprises a set of 6 complementary recesses 258 for engaging the protrusions 157, as shown in FIGS. 7b-c, where FIG. 7c is an axial cross-section through the subsystem 100.

FIGS. 8-11 show how each of the subsystems 100 of FIGS. 4-7 are movable between multiple configurations, i.e. how the baffle assembly 150 is engagable with respect to the subsystem 100 in at least one of a plurality of predetermined positions, which include fully open, fully closed and a least one intermediate open position, as the baffle assembly 150 is moved with respect to the air inlet 28, to alter air flow through the device e.g. resulting in different degrees of alignment between the baffle assembly aperture 154 and the air inlet 28.

In all of the example embodiments of FIGS. 4-11, the subsystem 100 comprises one (central) primary air inlet 28 and two secondary, bypass inlets 128, and the engagement mechanism provides 3 positive engagement positions for the baffle assembly 150 with respect to the air inlet 28. In these figures, the opening in the housing 12 defining the air inlet 28 is larger than the baffle assembly aperture 154. This enables the baffle assembly aperture 154 to align with the opening in the housing 12 in more than one position, as particularly highlighted in FIGS. 8a-8b and 10a-10b, so that multiple positions of the baffle assembly 150 are ‘open’ and permit airflow therethrough. Alternatively, or in addition, there may be multiple openings defining multiple primary air inlets 28 and/or multiple baffle assembly apertures 154 configured to align with the opening(s).

In particular, referring to FIG. 8a, a width y1 of the opening in the housing 28 defining the air inlet 28 may be at least 10%, 25%, 50%, 75% or 100% wider than the diameter y2 of the openings for the bypass air inlets 128 and/or the baffle assembly aperture 154. A length x1 of the opening in the housing 28 defining the air inlet 28 may be configured to enable the baffle assembly 150 to move between at least two engagement positions (e.g. corresponding to the fully open and partially open positions shown in FIGS. 8a and 8b), maintaining an air flow path through the air inlet 28 and selectively providing an air flow path through one or two of the bypass inlets 128. As indicated in FIG. 8a, a length x1 of the opening in the housing 12 defining the air inlet 28 may be equal to or greater than a distance x2 between the outermost edges of the bypass inlets 128 to provide such functionality.

FIGS. 8a-c show how the subsystem 100 of FIGS. 4a-c is movable between the plurality of predetermined positions. In FIGS. 8a-8c, the snap-fit projection 155 provides 3 positions for engaging with the spherical cap 257 on the housing 12, these being the lowest (end) points of the ramps 159 and the recess 158 in between.

In FIG. 8a, the subsystem is fully open, i.e. with all air inlets 28, 128 open; none covered or blocked, thereby providing minimal resistance to air being drawn into the system as the user inhales on the mouthpiece. Here, the housing protrusion 257 is engaged with the end point minimum of the right baffle assembly ramp 159.

In FIG. 8b, the subsystem is partially open, with the central air inlet 28 and the outermost bypass inlet 128 open, but the innermost bypass inlet 128 is covered by the baffle assembly 150. Here, the protrusion 257 is engaged with the hemispherical recess 158 in the snap-fit projection 155 of the slider 152.

In FIG. 8c, the subsystem is closed, with all air inlets 28, 128 covered by the baffle assembly 150. The closed configuration may effectively disable operation of the device (e.g. prevent power to the aerosol generator) and thus provide a child safety lock function. Here, the protrusion 257 is engaged with the end point minimum of the left baffle assembly ramp 159.

FIGS. 9a-c show how the subsystem 100 of FIGS. 5a-d is movable between the predetermined positions, akin to FIGS. 8a-c for the first particular embodiment. In FIG. 9a, the subsystem is fully open, corresponding to FIG. 8a for the first particular embodiment. Here, the housing protrusion 257 presses into the baffle assembly seal 156 as shown, but there is no recess 158 located in the seal 156 of FIG. 5d to provide a positive engagement feedback mechanism in this position.

Nevertheless, the user is reassured that the device is in the fully open position because they are unable to move the baffle assembly 150 further. By contrast, the alternative baffle assembly seal 156 of FIG. 5e provides a second, rightmost recess 158 in which the protrusion 257 would be received in this fully open position, thus giving a positive engagement feedback mechanism when the user moves the baffle assembly 150 into this position.

In FIG. 9b, the subsystem is partially open, with the central air inlet 28 and the outermost bypass inlet 128 open, but the innermost bypass inlet 128 is covered by the baffle assembly 150. Here, the complementary protrusion 257 in the housing 12 is engaged with the right-hand hemispherical recess 158 of the baffle assembly seal 156 of FIG. 5d, thus giving a positive engagement feedback mechanism when the user moves the assembly 150 into this position. Similarly, with the alternative baffle assembly seal 156 of FIG. 5e, the complementary protrusion 257 would engage the inner right-hand recess 158.

In FIG. 9c, the subsystem is closed, with all air inlets 28, 128 covered by the baffle assembly 150. Here, the protrusion 257 is engaged with the baffle aperture 154, thus again giving a positive engagement feedback mechanism when the user moves the assembly 150 into this position (and in the same manner with the baffle assembly seals 156 of FIGS. 5d and 5e). As noted above with respect to FIG. 5d, the left-hand recess 158 of FIG. 5d does not engage with any protrusion 257 and is simply to aid assembly.

FIGS. 10a-c show how the subsystem 100 of FIGS. 6a-c is movable between the predetermined positions, akin to FIGS. 8a-c for the first particular embodiment. In FIG. 10a, the subsystem is fully open. Here, the sensor seal complementary protrusion 257 abuts and engages an outer edge of the rightmost one of the projections 155.

In FIG. 10b, the subsystem is partially open, with the central air inlet 28 and the outermost bypass inlet 128 open, but the innermost bypass inlet 128 is covered by the baffle assembly 150. Here, the complementary protrusion 257 in the seal 130 is engaged in the recess 158 between the projections 155.

In FIG. 10c, the subsystem is closed, with all air inlets 28, 128 covered by the baffle assembly 150. Here, the complementary protrusion 257 in the seal 130 engages an outer edge of the leftmost of the projections 155.

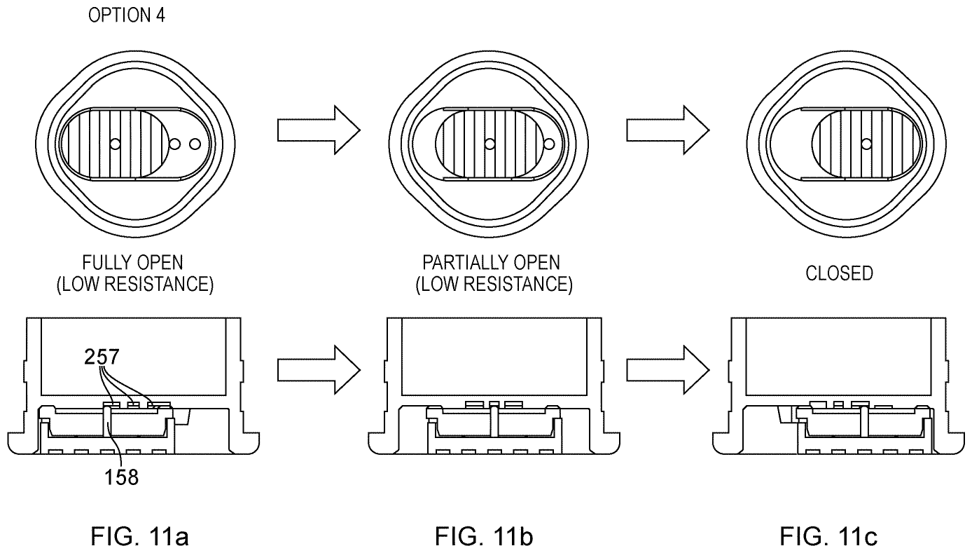

FIGS. 11a-c show how the subsystem 100 of FIGS. 7a-c is movable between the predetermined positions, akin to FIGS. 8a-c for the first particular embodiment. In FIG. 11a, the subsystem is fully open. Here, the pair of baffle assembly protrusions 158 engage the first pair of complementary recesses 257 in the housing 12, furthest from the bypass inlets 128 (not shown in FIG. 11a).

In FIG. 11b, the subsystem is partially open, with the central air inlet 28 and the outermost bypass inlet 128 (not shown) open, but the innermost bypass inlet 128 (not shown) is covered by the baffle assembly 150. Here, the pair of baffle assembly protrusions 158 engage the intermediate pair of complementary recesses 257.

In FIG. 11c, the subsystem is closed, with all air inlets 28, 128 covered by the baffle assembly 150. Here, the pair of baffle protrusions 158 engage the pair of complementary recesses 257 closest to the bypass inlets 128 (not shown).

Generally, in some embodiments, the air inlets 28, 128 comprise one or more primary air inlets 28 and one or more secondary, bypass air inlets 128 and the plurality of predetermined positions comprise:
a. a first position wherein the baffle assembly aperture 154 is not aligned with any of the primary air inlets 28, optionally wherein the first position comprises a closed position wherein none of the air inlets 28, 128 provide an air flow path therethrough;
b. a second, intermediate position wherein the baffle assembly aperture 154 is aligned with at least one of the primary air inlets 28 and optionally one or more of the bypass air inlets 128 provide an air flow path therethrough; and
C. a third, fully open position, wherein the baffle assembly aperture 154 is aligned with at least one of the primary air inlets and all bypass air inlets provide an air flow path therethrough.

In some embodiments, one or more protrusions 157, 257, recesses 158, 258 and/or ramps 159, 259 are described or depicted. In embodiments, any number of protrusions and/or recesses may be provided, particularly in pairs on opposing sides of the relevant component, e.g. to provide constraint in multiple dimensions.

In some embodiments, the subsystem comprises a housing 12 having an opening defining the air inlet(s); and/or a housing having a baffle assembly cavity 135 configured to receive the baffle assembly 150; and/or a housing having a power supply cavity 126 configured to receive a power supply 26. In some such embodiments, the baffle assembly cavity 135 or the power supply cavity 126 may comprise the complementary protrusion 257 or recess 258 for engaging with the baffle assembly protrusion 157 or recess 258.

In some embodiments, the subsystem 100 comprises multiple air inlets 28, 128 and/or multiple apertures 154 in the baffle assembly 150. In some embodiments, different ones of the plurality of predetermined positions of the baffle assembly 150 result in different degrees of alignment between the single or multiple baffle apertures 154 and the single or multiple air inlets 28, 128, to alter air flow through the air inlet(s) 28, 128. In some embodiments, there are multiple air inlets 28, 128 wherein movement of the baffle assembly 150 alters air flow through one or more of the multiple air inlets 28, 128.

In some embodiments, an axis of extent of the baffle assembly aperture 154 is substantially parallel to a straight flow path between the air inlet 28 and an outlet 50, minimising disruption by the baffle assembly 150 to the air flow through the system.

In some embodiments, the plurality of predetermined positions comprise three or more positions, or consist of a plurality of positions, for the baffle assembly aperture 154, the aperture positions forming a substantially straight line. In some embodiments, e.g. as shown in FIGS. 8-11, the plurality of predetermined positions provide minimal or zero alignment, partial alignment and maximum or complete alignment between the baffle assembly aperture 154 and the air inlet 28, sequentially.

In some embodiments, the plurality of predetermined positions define a range of movement of the baffle assembly 150 with respect to the subsystem 100, and the subsystem 100 is configured to prevent movement of the baffle assembly 150 beyond said range, e.g. by the subsystem 100 or the baffle assembly 150 comprising a shoulder for abutting against the other of the subsystem 100 or baffle assembly 150 at each end of the range. In some embodiments, the baffle assembly 150 moves by translation (e.g. sliding). In further embodiments, the baffle assembly 150 is rotatable.

In some embodiments, different ones of the plurality of predetermined positions provide a flow path through different numbers of multiple air inlets 28, 128. In some embodiments, different ones of the plurality of predetermined positions provide alignment between different numbers of multiple primary air inlets 28 with the baffle assembly aperture(s) 154.

In some embodiments, extreme positions of the plurality of predetermined positions are spaced within the range of 1-50 mm, 2-40 mm, 3-30 mm, 4-20 mm or substantially 5-10 mm apart. In some embodiments, adjacent positions of the plurality of predetermined positions are within the range of 0.5-5 mm, 1-3 mm or substantially 2 mm apart.

### Baffle Assembly With No Aperture

In further embodiments (not specifically shown), the baffle assembly 150 does not comprise an aperture 154. Nevertheless, the baffle assembly 150 remains movable to allow different amounts of air flow into the system-the baffle assembly 150 is movable to cover or expose the air inlet(s) 28, 128, simply without aligning the baffle aperture 154 with one or more of the inlet(s) 28, 128.

For example, the baffle assembly 150 may be slidable between the closed, partially open and fully open positions shown in FIGS. 8-11, just without a baffle aperture 154. For example, referring to the end-on closed position in FIG. 11, the baffle assembly 150 without aperture 154 covers/blocks all inlets 28, 128. In partially open positions, the baffle assembly 150 may partially or fully expose a primary inlet 28 (e.g. the rightmost exposed inlet in the end-on view of FIG. 11b), and may optionally partially or fully expose one or more additional inlets 28, 128, e.g. as the baffle assembly 150 slides from the closed position towards the open position. In a fully open position, the baffle assembly 150 exposes all inlets 28, 128 (e.g. the additionally exposed inlet in the end-on view of FIG. 11a). Such a baffle assembly 150 without an aperture 154 thus can provide the same functionality as in FIGS. 8-11, with the baffle assembly 150 having different positions providing different degrees of opening of the air inlet(s) 28, to alter air flow therethrough, but will generally provide a less compact arrangement (since the baffle assembly 150 must be moved e.g. translated wholly away from all inlets 28, 128 to expose them fully).

Accordingly, the present invention may provide an aerosol delivery subsystem 100 for adjusting fluid flow in an aerosol delivery system 1, comprising:
an air inlet 28;
a baffle assembly 150; and
an engagement mechanism for positively engaging the baffle assembly 150 with respect to the subsystem 100 in at least one of a plurality of predetermined positions as the baffle assembly 150 is moved with respect to the air inlet 28, wherein different ones of the plurality of predetermined positions result in different degrees of opening of the air inlet(s) 28, to alter air flow through the air inlet 28.

The above arrangement may be combined with any other features disclosed herein-such combinations are specifically contemplated.

### Baffle Assembly With No Engagement Mechanism

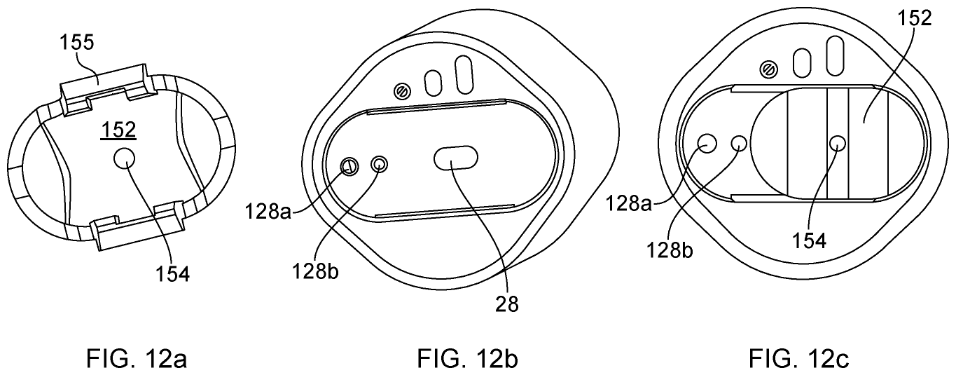

FIGS. 12a-c are schematic perspective and end views of an aerosol delivery subsystem in accordance with a fifth particular embodiment of the disclosure, highlighting different aspects of further embodiments. By contrast to the embodiments of FIGS. 4-11, the embodiment of FIGS. 12a-c does not comprise an engagement mechanism for positively engaging the baffle assembly 150 with respect to the subsystem 100 in at least one of a plurality of predetermined positions. Here, the baffle assembly 150 may be freely movable between the fully open and fully closed positions, without a discrete intermediate open position in which the baffle assembly 150 can consistently be engaged via the engagement mechanism.

FIG. 12a illustrates the slider 152 of the baffle assembly 150, which comprises the slider 152 and a baffle assembly seal 156 and is similar to prior embodiments, but without an engagement mechanism. The slider 152 comprises an aperture 154 therethrough and two snap-fit projections 155 in the form of cantilever projections on opposing long sides of the slider 152, for securing the baffle assembly 150 within the subsystem 100, e.g. to the housing 12. The slider 152 is sized and shaped to be operable by a user's fingers or thumb from a base of the system by being discorectangular in shape (a stadium) and comprising curved recesses in the centre and at each axial end thereof (see FIG. 12b). These features are designed to be operated by the user's digits thus increase usability of the baffle assembly 150, making the slider 152 easier to move.

FIG. 12b shows a perspective end view of the subsystem 100 without the baffle assembly 150. As shown, the housing 12 has 3 aligned openings: one elongate oval opening defining a primary air inlet 28 as well as two side, secondary air inlets 128. As shown, a first, outermost secondary air inlet 128a has a larger opening diameter than an innermost secondary air inlet 128b. The air inlet openings 28, 128 may generally be different sizes and/or may be substantially within the range 0.5-5.0 mm, 0.5-3.0 mm, 0.5-2.5 mm or 1.0-2.0 mm diameter. In the arrangement shown in FIGS. 12 a-c, the innermost secondary inlet 128 is the last to be exposed as the slider 152 moves from a closed position (on the left), covering all inlets 28, 128, to the fully open position on the right, as depicted in FIG. 12c, and such progressively smaller secondary inlets 128 allow fine tuning of the draw resistance for the user. Additional secondary air inlets 128 may be provided, preferably decreasing in diameter in the direction of the slider 152 as it moves towards the fully open position.

The subsystem 100 comprises a fluid flow sensor 30 (not shown) and the primary air inlet 28 (which is illustrated as elongate but may alternatively comprise multiple inlets 28) provides an air flow path into the subsystem 100 via the fluid flow sensor 30, whilst the secondary air inlets 128 provide air flow paths into the subsystem 100 that bypass the fluid flow sensor 30. Such an arrangement is beneficial since the primary air inlet 28 can be used to provide air flow to the sensor 30 and thus can be used to reliably trigger operation of the device (e.g. activating the aerosol generator) in use and equally deactivate the device when the primary air inlet 28 is closed, acting as a safety feature, whilst the secondary air inlets 128 allow adjustment of the draw resistance, i.e. provide variable ventilation, without complicating the air flow paths through the system or requiring multiple flow sensors 30. By contrast, if all inlets 28, 128 provide an air flow path into the subsystem 100 via the fluid flow sensor 30, then this can lead to a less compact design and/or unreliable triggering of the flow sensor 30.

FIG. 12c shows the assembled subsystem 100 with the baffle assembly 150 installed therein. The baffle assembly 150 is movable with respect to the subsystem 100 into a plurality of positions to selectively alter air flow through one or more of the primary and secondary air inlets 28, 128. In particular, the baffle assembly 150 is movable to selectively cover the primary air inlet 28 and the secondary air inlets 128.

In FIG. 12c, the baffle assembly 150 is in a fully open position, where the baffle assembly aperture 154 aligns with a rightmost end of the elongate primary air inlet 28, and the baffle assembly 150 does not cover either of the secondary air inlets 128-these are exposed/unobstructed, thus allowing maximum air flow into the system, minimising the pressure drop in use. The baffle assembly 150 can be moved from the fully open position of FIG. 12c to a partially open position by sliding the baffle assembly 150 to the left, to partially or fully cover the smaller, inner secondary air inlet 128b, reducing the air flow into the system. The primary air inlet 28 remains uncovered/open since the baffle assembly aperture 154 remains aligned with the elongate inlet 28 (e.g. aligned with the middle of the elongate inlet 28). When moving further left, the baffle assembly 150 may fully cover the innermost secondary air inlet 128b and partially or fully cover the outermost secondary air inlet 128a, reducing air flow into the system further. Preferably, there is a minimal open position where the primary air inlet 28 is at least partially but preferably fully open/uncovered, whilst all secondary air inlets 128 are covered.

The user may thus move the baffle assembly 150 to progressively expose more of the air inlet openings to allow more air flow into the subsystem 100 in use. Accordingly the plurality of positions may comprise a first (closed) position wherein the baffle assembly 150 covers at least the primary air inlet 28, optionally all of the inlets 28, 128; a second (intermediate open) position wherein the baffle assembly 150 uncovers the primary air inlet 28; a third (intermediate open) position wherein the baffle assembly 150 uncovers both the primary air inlet 28 and a secondary air inlet 128; and a fourth (fully open) position in which the baffle assembly 150 uncovers the primary air inlet 28 and all of multiple secondary air inlets 128.

The subsystem may comprise a sensor seal 130 having a cavity for receiving/locating the sensor 30 in the subsystem and providing an air flow path thereto. Preferably, the fluid flow sensor 30 is located proximal or adjacent to at least one of the air inlets 28 and/or a sensing portion of the fluid flow sensor 30 is directly exposed to or within a fluid flow path from the air inlet 28 towards an aerosol generator 48.

In the embodiment of FIGS. 12a-c, a single (elongate) primary air inlet 28 is provided, which provides an air flow path into the subsystem 100 via a single fluid flow sensor 30. In further embodiments, additional primary air inlets 28 may be provided which provide an air flow path into the subsystem 100 via the single fluid flow sensor 30, or additional fluid flow sensors may be provided. For example, multiple discrete primary air inlets 28 may be provided instead of a single elongate inlet 28. Similarly, a single baffle assembly aperture 154 is illustrated-in further embodiments, the baffle assembly 150 comprises no aperture or multiple apertures 154.

Although the secondary air inlet openings 128 are illustrated as circular, these and all other openings may be of any shape. In particular, the secondary air inlet openings 128 may be elongate in the axial direction of movement of the baffle assembly 150, akin to the primary inlet 28, which would effectively provide continuously variable adjustment within a predetermined range.

The arrangement of FIGS. 12a-c may be combined with any other features disclosed herein-such combinations are specifically contemplated. In particular, although the embodiment of FIGS. 12a-c is not shown to comprise an engagement mechanism, in further embodiments, the subsystem comprises an engagement mechanism, e.g. for positively engaging the baffle assembly 150 with respect to the subsystem 100 in at least one of the positions, preferably in a closed position (providing a safety lock feature) and/or one or more partially-open configurations, to thus define a predetermined, consistently engagable partially-open position.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention.

Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future. Protection may also be sought for any features disclosed in any one or more published documents referenced herein in combination with the present disclosure.

### INDEX TO REFERENCE NUMERALS

1 aerosol delivery system
2 reusable part
4 cartridge part
6 Interface between reusable part and cartridge part
12 reusable part housing
14, 16 user input buttons
20 user programming circuitry
22 controller
24 display
26 power source
28 air inlet
30 fluid flow sensor
31 printed circuit board (PCB)
32 sensor cavity or chamber
34 chamber wall
42 cartridge housing
44 chamber or reservoir
46 wick
48 aerosol generator
50 mouthpiece outlet
51 airflow path through the reusable part
52 reusable part airflow path
60 mouthpiece housing
100 aerosol delivery subsystem
126 power supply cavity
128 bypass air inlet
130 sensor seal
135 baffle assembly cavity
150 baffle assembly
152 baffle assembly slider
154 baffle assembly aperture
155 baffle assembly snap-fit projection
156 baffle assembly seal
157 baffle assembly protrusion
158 baffle assembly recess
159 baffle assembly ramp
257 complementary protrusion
258 complementary recess
259 complementary ramp

Any reference numerals in the claims are purely to enhance clarity and do not impact the scope of protection in any way.

### Representative Features

1. An aerosol delivery subsystem 100 for adjusting fluid flow in an aerosol delivery system 1, comprising:
   a. a primary air inlet 28 into the subsystem 100;
   b. a secondary air inlet 128 into the subsystem 100;
   c. a fluid flow sensor 30; and
   d. a baffle assembly 150, wherein:
      i. the primary air inlet 28 provides an air flow path into the subsystem 100 via the fluid flow sensor 30;
   ii. the secondary air inlet 128 provides an air flow path into the subsystem 100 bypassing the fluid flow sensor 30; and
   iii. the baffle assembly 150 is movable with respect to the subsystem 100 into a plurality of positions to selectively alter air flow through one or more of the primary and secondary air inlets 28, 128.
2. The subsystem of clause 1, wherein the baffle assembly 150 is movable with respect to the subsystem 100 to selectively cover the primary air inlet 28 and the secondary air inlet 128.
3. The subsystem of any preceding clause, wherein the plurality of positions comprise:
   a. a first position, wherein the baffle assembly 150 covers at least the primary air inlet 28;
   b. a second position, wherein the baffle assembly 150 uncovers the primary air inlet 28; and
   c. a third position, wherein the baffle assembly 150 uncovers both the primary air inlet 28 and the secondary air inlet 128.
4. The subsystem of any preceding clause, wherein the plurality of positions comprise:
   a. a position in which the baffle assembly 150 uncovers the primary air inlet 28 and multiple secondary air inlets 128.
5. The subsystem of any preceding clause, wherein the primary and/or secondary air inlet openings 28, 128 are substantially within the range 0.5-5.0 mm, 0.5-3.0 mm, 0.5-2.5 mm or 1.0-2.0 mm diameter.
6. The subsystem of any preceding clause, wherein the primary and secondary air inlet openings 28, 128 are of different sizes.
7. The subsystem of any preceding clause, wherein at least two of the secondary air inlets 128 have differently-sized openings.
8. The subsystem of clause 7, wherein an outer secondary air inlet 128 is larger in diameter than an inner secondary air inlet 128.
9. The subsystem of any preceding clause, wherein the subsystem comprises only a single fluid flow sensor 30.
10. The subsystem of any preceding clause, comprising an engagement mechanism for positively engaging the baffle assembly 150 with respect to the subsystem 100 in at least one of the positions.
11. The subsystem of clause 10, wherein the engagement mechanism comprises one or more engaging or interlocking pairs of protrusions and recesses.
12. The subsystem of clause, 10 or 11 wherein:
   a. the engagement mechanism comprises the baffle assembly 150 having a protrusion 157 or a recess 158 and the subsystem 100 having a complementary protrusion 257 or recess 258, for engaging with the baffle assembly protrusion 157 or recess 158; and/or
   b. the engagement mechanism is for positively engaging the baffle assembly 150 with respect to the subsystem 100 in at least one intermediate open position of the baffle assembly 150 with respect to the air inlets 28, 128.
13. The subsystem of clause 10, 11 or 12, wherein:
   one or more of the protrusions 157, 257 and/or recesses 158, 258 comprises a spherical cap or hemisphere; and/or
   the engagement mechanism comprises a ramp 159, 259 for guiding and/or biasing the baffle assembly 150 into one or more of the plurality of positions.
14. The subsystem of any preceding clause, wherein the baffle assembly 150 comprises one or more snap-fit projections 155 for securing the baffle assembly 150 within the subsystem 100.
15. The subsystem of clause 14, wherein the baffle snap-fit projection 155 comprises:
   a. one or more cantilever, torsional and/or annular snap-fit projections 155; and/or
   b. the baffle assembly protrusion 157 or recess 158; and/or
   c. the ramp 159.
16. The subsystem of any preceding clause, wherein the baffle assembly 150 comprises a baffle assembly seal 156, optionally wherein the baffle assembly seal 156 comprises the baffle assembly protrusion 157 or recess 158.
17. The subsystem of any preceding clause, configured to:
   a. enable a supply of power to an aerosol generator 48 when one or more of the primary air inlets 28are open and provide an air flow path therethrough; and/or
   b. adjust a supply of power to an aerosol generator 48 when moving between positions; and/or
   c. disable a supply of power to an aerosol generator 48 when at least the primary air inlet(s) 28 is/are closed.
18. The subsystem of any preceding clause, further comprising:
   a. a housing 12 having an opening defining the primary and/or secondary air inlets 28, 128; and/or
   b. a housing 12 having a baffle assembly cavity 135 configured to receive the baffle assembly 150; and/or
   c. a housing 12 having a power supply cavity 126 configured to receive a power supply 26; and/or
   d. a sensor seal 130 for receiving the sensor 30.
19. The subsystem of clause 18, wherein the baffle assembly cavity 135, the power supply cavity 126, or the sensor seal 130 comprises the complementary protrusion 257 or recess 258 for engaging with the baffle assembly protrusion 157 or recess 158.
20. The subsystem of any preceding clause, comprising:
   a. a sensor seal 130 having a cavity for receiving the fluid flow sensor 30 proximal or adjacent to the primary air inlet 28; and/or
   b. the fluid flow sensor 30 proximal or adjacent to the primary air inlet 28; and/or
   c. the subsystem 100 being configured to activate an aerosol generator 48 in response to the fluid flow sensor 30 sensing fluid flow, and/or
   d. a sensing portion of the fluid flow sensor 30 directly exposed to or within a fluid flow path from the primary air inlet 28 towards an aerosol generator 48.
21. The subsystem of any preceding clause, wherein:
   a. the primary air inlet(s) 28 to the fluid flow sensor 30 are configured to deliver air to an aerosol generator 48 in use; and/or
   b. the secondary air inlet(s) 128 are configured to deliver air to an aerosol generator 48 in use; and/or
   c. the secondary air inlet(s) 128 are configured to provide airflow through the system, bypassing the aerosol generator 48 in use.
22. The subsystem of any preceding clause, wherein the baffle assembly 150 has:
   a. one or more surface features; and/or
   b. one or more curved recesses; and/or
   c. a plurality of alternating ridges and recesses extending perpendicular to an axis of movement of the baffle between the predetermined positions; and/or
   d. a length of about 5-50 mm; and/or
   e. a width of about 2-25 mm; and/or
   f. a discorectangular shape.
23. The subsystem of any preceding clause, wherein the baffle assembly 150 has an aperture therethrough and:
   a. the plurality of positions comprise three or more positions for the baffle assembly aperture 154, the aperture positions forming a substantially straight line; and/or
   b. the plurality of positions consist of a plurality of positions for the baffle assembly aperture 154, the aperture positions forming a substantially straight line; and/or
   c. the plurality of positions provide minimal or zero alignment, partial alignment and maximum or complete alignment between the baffle assembly aperture 154 and the primary air inlet 28, sequentially; and/or
d. the plurality of positions define a range of movement of the baffle assembly 150 with respect to the subsystem 100, and the subsystem 100 is configured to prevent movement of the baffle assembly 150 beyond said range, preferably by the subsystem 100 or the baffle assembly 150 comprising a shoulder for abutting against the other of the subsystem 100 or baffle assembly 150 at each end of the range; and/or
e. different ones of the plurality of positions provide a flow path through different numbers of the air inlets 28, 128; and/or
f. different ones of the plurality of positions provide alignment between different numbers of multiple primary air inlets 28 with the baffle assembly aperture(s) 154; and/or
g. extreme positions of the plurality of positions are spaced within the range of 1-50 mm, 2-40 mm, 3-30 mm, 4-20 mm or substantially 5-10 mm apart; and/or
h. adjacent positions of the plurality of positions are within the range of 0.5-5 mm, 1-3 mm or substantially 2 mm apart.
24. The subsystem of any preceding clause, wherein the plurality of positions comprise:
   a. a first position wherein the baffle assembly aperture 154 is not aligned with the or any of the primary air inlet(s) 28, optionally wherein the first position comprises a closed position wherein none of the primary and optionally none of the secondary air inlets 28, 128 provide an air flow path therethrough;
   b. a second, intermediate position wherein the baffle assembly aperture 154 is aligned with at least one of the primary air inlet(s) 28; and optionally one or more of the secondary air inlet(s) 128 provide an air flow path therethrough; and
   c. a third, fully open position, wherein the baffle assembly aperture 154 is aligned with at least one primary air inlet(s) 28 and all secondary air inlets 128 provide an air flow path therethrough.
25. The subsystem of any preceding clause, or an aerosol delivery system comprising the subsystem of any preceding clause, further comprising:
   a. an aerosol generator; and/or
   b. a cartridge or cartomizer housing an aerosol-generating material for generating aerosol for inhalation by a user; and/or
   c. a mouthpiece; and/or
   d. a controller; and/or
   e. a power source.
26. A method of manufacturing an aerosol delivery subsystem for an aerosol delivery system, comprising providing:
   a. a primary air inlet 28;
B. a Secondary Air Inlet 128;
   c. a fluid flow sensor 30; and
   d. a baffle assembly 150, the method comprising configuring the subsystem wherein:
      i. the primary air inlet 28 provides an air flow path into the subsystem 100 via the fluid flow sensor 30;
      ii. the secondary air inlet 128 provides an air flow path into the subsystem 100 bypassing the fluid flow sensor 30; and
      iii. the baffle assembly 150 is movable with respect to the subsystem 100 into a plurality of positions to selectively alter air flow through one or more of the primary and secondary air inlets 28, 128.
27. An aerosol delivery subsystem 100 for adjusting fluid flow in an aerosol delivery system 1, comprising:
   a. a primary air inlet means 28;
   b. a secondary air inlet means 128;
   c. a fluid flow sensing means 30; and
   d. a baffle assembly means 150, wherein:
      i. the primary air inlet means 28 provides an air flow path into the subsystem 100 via the fluid flow sensor 30;
      ii. the secondary air inlet means 128 provides an air flow path into the subsystem 100 bypassing the fluid flow sensor 30; and
      iii. the baffle assembly 150 means is movable with respect to the subsystem 100 into a plurality of positions to selectively alter air flow through one or more of the primary and secondary air inlet means 28, 128.

1. An aerosol delivery subsystem for adjusting fluid flow in an aerosol delivery system, comprising:
a. an air inlet;
b. a baffle assembly having an aperture therethrough; and
c. an engagement mechanism for positively engaging the baffle assembly with respect to the subsystem in at least one of a plurality of predetermined positions as the baffle assembly is moved with respect to the air inlet, wherein different ones of the plurality of predetermined positions result in different degrees of alignment between the baffle assembly aperture and the air inlet, to alter air flow through the air inlet.

2. (canceled)

3. The subsystem of claim 1, wherein:
a) engagement mechanism comprises one or more engaging or interlocking pairs of protrusions and recesses; and/or
b) the engagement mechanism comprises the baffle assembly having a protrusion or a recess and the subsystem having a complementary protrusion or recess, for engaging with the baffle assembly protrusion or recess; and/or
c) the engagement mechanism is for positively engaging the baffle assembly with respect to the subsystem in at least one intermediate open position of the baffle assembly with respect to the air inlet(s).

4. The subsystem of claim 3, wherein one or more of the protrusions and/or recesses comprises a spherical cap or hemisphere.

5. The subsystem of claim 1, wherein the engagement mechanism comprises a ramp for guiding and/or biasing the baffle assembly into one or more of the plurality of predetermined positions.

6. The subsystem of claim 1, wherein the baffle assembly comprises one or more snap-fit projections for securing the baffle assembly within the subsystem; and/or wherein the baffle assembly comprises a baffle assembly seal, optionally wherein the baffle assembly seal comprises the baffle assembly protrusion or recess.

7. The subsystem of claim 6, wherein the baffle snap-fit projection comprises:
a. one or more cantilever, torsional and/or annular snap-fit projections; and/or
b. the baffle assembly protrusion or recess; and/or
c. the ramp.

8. (canceled)

9. The subsystem of claim 1, configured to:
a) enable a supply of power to an aerosol generator when one or more of the air inlets are open and provide an air flow path therethrough; and/or
b) adjust a supply of power to an aerosol generator when moving between predetermined positions providing non-zero degrees of alignment between the baffle assembly aperture and the air inlets; and/or
c) disable a supply of power to an aerosol generator when all air inlets are closed.

10. The subsystem of claim 1, further comprising:
a) a housing having an opening defining the air inlet; and/or
b) a housing having a baffle assembly cavity configured to receive the baffle assembly; and/or
c) a housing having a power supply cavity configured to receive a power supply; and/or
d) a sensor seal for receiving a sensor;
e) optionally wherein the baffle assembly cavity, the power supply cavity, or the sensor seal comprises the complementary protrusion or recess for engaging with the baffle assembly protrusion or recess.

11. (canceled)

12. The subsystem of claim 1, comprising:
a) multiple air inlets and/or multiple apertures in the baffle assembly; or
b) multiple air inlets wherein movement of the baffle assembly alters air flow through one or more of the multiple air inlets; or
c) multiple air inlets and multiple apertures in the baffle assembly, wherein different ones of the plurality of predetermined positions of the baffle assembly result in different degrees of alignment between the multiple baffle apertures and the multiple air inlets to alter air flow through the air inlets.

13. The subsystem of claim 1, comprising:
a) a sensor seal having a cavity for receiving a fluid flow sensor proximal or adjacent to the air inlet; and/or
b) a fluid flow sensor proximal or adjacent to the air inlet; and/or
c) a fluid flow sensor, wherein the subsystem is configured to activate an aerosol generator in response to sensing fluid flow; and/or
d) a fluid flow sensor, wherein a sensing portion of the fluid flow sensor is directly exposed to or within a fluid flow path from the air inlet towards an aerosol generator.

14. The subsystem of claim 13, comprising the fluid flow sensor and multiple air inlets, wherein the multiple air inlets comprise one or more primary air inlets to the fluid flow sensor and one or more secondary, bypass air inlets bypassing the fluid flow sensor, optionally wherein:
a) the primary air inlets to the fluid flow sensor are configured to deliver air to an aerosol generator in use; and/or
b) the secondary, bypass air inlets are configured to deliver air to an aerosol generator in use; and/or
c) the secondary, bypass air inlets are configured to provide airflow through the system, bypassing the aerosol generator in use.

15. The subsystem of claim 1, wherein an axis of extent of the baffle assembly aperture is substantially parallel to a straight flow path between the air inlet and an outlet.

16. The subsystem of claim 1, wherein the baffle assembly has:
a) one or more surface features; and/or
b) a plurality of alternating ridges and recesses extending perpendicular to an axis of movement of the baffle between the predetermined positions; and/or
c) a length of about 5-50 mm; and/or
d) a width of about 2-25 mm; and/or
e) a discorectangular shape.

17. The subsystem of claim 1, wherein:
a) the plurality of predetermined positions comprise three or more positions for the baffle assembly aperture, the aperture positions forming a substantially straight line; and/or
b) the plurality of predetermined positions consist of a plurality of positions for the baffle assembly aperture, the aperture positions forming a substantially straight line; and/or
c) the plurality of predetermined positions provide minimal or zero alignment, partial alignment and maximum or complete alignment between the baffle assembly aperture and the air inlet, sequentially; and/or
d) the plurality of predetermined positions define a range of movement of the baffle assembly with respect to the subsystem, and the subsystem is configured to prevent movement of the baffle assembly beyond said range, preferably by the subsystem or the baffle assembly comprising a shoulder for abutting against the other of the subsystem or baffle assembly at each end of the range; and/or
e) different ones of the plurality of predetermined positions provide a flow path through different numbers of multiple air inlets; and/or
f) different ones of the plurality of predetermined positions provide alignment between different numbers of multiple air inlets with the baffle assembly aperture(s); and/or
g) extreme positions of the plurality of predetermined positions are spaced within the range of 1-50 mm, 2-40 mm, 3-30 mm, 4-20 mm or substantially 5-10 mm apart; and/or
h) adjacent positions of the plurality of predetermined positions are within the range of 0.5-5 mm, 1-3 mm or substantially 2 mm apart.

18. The subsystem of claim 1, wherein the air inlets comprise one or more primary air inlets and one or more secondary, bypass air inlets and the plurality of predetermined positions comprise:
a. a first position wherein the baffle assembly aperture is not aligned with any of the primary air inlets, optionally wherein the first position comprises a closed position wherein none of the air inlets provide an air flow path therethrough;
b) a second, intermediate position wherein the baffle assembly aperture is aligned with at least one of the primary air inlets; and optionally one or more of the bypass air inlets provide an air flow path therethrough; and
c) a third, fully open position, wherein the baffle assembly aperture is aligned with at least one of the primary air inlets and all bypass air inlets provide an air flow path therethrough.

19. The subsystem of claim 1, or an aerosol delivery system comprising the subsystem of any preceding claim, further comprising:
a) an aerosol generator; and/or
b) a cartridge or cartomizer housing an aerosol-generating material for generating aerosol for inhalation by a user; and/or
c) a mouthpiece; and/or
d) a controller; and/or
e) a power source.

20. The aerosol delivery subsystem of claim 3, further comprising a housing configured to receive the baffle assembly, wherein:
a. the baffle assembly comprises a baffle slider having a snap-fit projection with a hemispherical baffle assembly recess therein and a ramp for guiding the baffle assembly into at least one of the plurality of predetermined positions; and
b. the housing comprises a complementary spherical cap protrusion for positively engaging with the recess on the baffle assembly; or
c. the baffle assembly comprises a seal, the seal comprising a hemispherical baffle assembly recess; and
d. the housing comprises a complementary spherical cap protrusion for positively engaging with the baffle assembly recess on the seal.

21. (canceled)

22. The aerosol delivery subsystem of claim 3, further comprising a sensor for detecting fluid flow and a sensor seal for sealing around the sensor, wherein:
the baffle assembly comprises a baffle slider having a snap-fit projection, the snap-fit projection having the baffle assembly recess therein; and
the sensor seal comprises the complementary projection; or further comprising a housing configured to receive the baffle assembly, wherein:
the baffle assembly comprises a baffle slider having the baffle assembly protrusion; and
the housing comprises the complementary recess.

23. (canceled)

24. A method of manufacturing an aerosol delivery subsystem for an aerosol delivery system, comprising providing:
a) an air inlet;
b) a baffle assembly having an aperture therethrough; and
c) an engagement mechanism;
the method comprising configuring the engagement mechanism for positively engaging the baffle assembly with respect to the subsystem in at least one of a plurality of predetermined positions as the baffle assembly is moved with respect to the air inlet, wherein different ones of the plurality of predetermined positions result in different degrees of alignment between the baffle assembly aperture and the air inlet, to alter air flow through the air inlet.

25. An aerosol delivery subsystem for adjusting fluid flow in an aerosol delivery system, comprising:
a) an air inlet means;
b) a baffle assembly means having an aperture therethrough; and
c) an engagement means for positively engaging the baffle assembly with respect to the subsystem in at least one of a plurality of predetermined positions as the baffle assembly is moved with respect to the air inlet, wherein different ones of the plurality of predetermined positions result in different degrees of alignment between the baffle assembly aperture and the air inlet, to alter air flow through the air inlet.
